(19) United States
(12) Patent Application Publication
(10) Pub. No.: US 2003/0096403 A1
(43) Pub. Date: May 22, 2003

(54) Humanized antibody to surface antigen s of hepatitis b virus and a preparing method thereof

(76) Inventors: Hyo-Jeong Hong, Taejeon-si (KR); Keun-Soo Kim, Kyunggi-do (KR)

Correspondence Address: GATES & COOPER LLP, HOWARD HUGHES CENTER, 6701 CENTER DRIVE WEST, SUITE 1050, LOS ANGELES, CA, 90045, US

(21) Appl. No.: 10/148,844

(22) PCT Filed: Oct. 4, 2001
(86) PCT No.: PCT/KR01/01657

(51) Int. Cl. C12Q 1/68; C12N 5/06; C12N 5/16; G01N 33/53

(52) U.S. Cl. 435/345; 435/332; 435/6; 436/548

(57) ABSTRACT

The present invention relates to the humanized antibodies to surface antigen S of hepatitis B virus and a preparing method thereof. Particularly, it relates to the humanized antibodies which comprise heavy and light chains having amino acid sequences originated from human antibodies at the HCDR1, HCDR2, HCDR3 and LCDR1, LCDR2, LCDR3 of their variable regions, expression vectors containing each of the heavy and light chain genes of the humanized antibody and transformant which can produce humanized antibody by transfection with heavy and light chain expression vectors and a preparing method thereof. A humanized antibody of the present invention is more humanized than that of the previous arts. So, it minimizes the probability of immune response in humans and has good antigen binding capacity, making it a excellent candidate for prevention and treatment of the hepatitis B virus infection.

### FIELD OF THE INVENTION

The present invention relates to the humanized antibodies to surface antigen S of hepatitis B virus and a preparing method thereof. Particularly, it relates to the humanized antibodies which comprise heavy and light chains having amino acid sequences originated from human antibodies at the HCDR1, HCDR2, HCDR3 and LCDR1, LCDR2, LCDR3 of their variable regions, minimizing human anti-mouse antibody (HAMA) response by eliminating peptide sequences from heavy chain of above humanized antibody which bind MHC class II molecules, expression vectors containing each of the heavy and light chain genes of the humanized antibody and transformant which can produce humanized antibody by transfection with heavy and light chain expression vectors and a mass-preparation method of humanized antibody by culturing above transformant.

### BACKGROUND OF THE INVENTION

Hepatitis B virus (HBV) invades into human body and causes chronic and acute hepatitis. If the condition grows worse, it can be a pathogen causing cirrhosis and cancer of the liver. It is estimated that up to three hundred millions patients in the world are infected with HBV (Tiollais & Buendia, Sci. Am., 264, 48, 1991). The envelope of HBV is composed of 3 types of proteins. Specifically, it is composed of major protein comprising S antigen, middle protein comprising S antigen and pre-S2 antigen, and large protein comprising S antigen, pre-S2 antigen and pre-S1 antigen (Neurath, A. R. and Kent, S. B. H., Adv. Vir. Res., 34: 65-142, 1988). All of the above-mentioned HBV surface antigen proteins can induce antibodies which neutralize HBV. Among them, S antigen takes part of about 80% of the total envelop protein and has neutralizing epitope called “Common a” which is commonly present in all of HBV subtypes.

When infected with HBV, for example, in cases of new-born babies born from HBV positive mother or medical institution employees exposed to HBV or recipients undergoing liver transplantation from patient having HBV-related liver diseases, HB immune globulin (HBIG) has been used to prevent the HBV infection (Beasley et al., Lancet, 2, 1099, 1983; Todo et al., Hepatol., 13, 619, 1991). However, since the currently used HBIG is prepared from human plasma, it has the disadvantages that the specificity against antigen is low and the probability of contamination is relatively high. Moreover, human blood has to be continuously supplied.

In order to overcome these disadvantages, method using mouse monoclonal antibody against HBV surface antigen has been developed. Generally, mouse monoclonal antibody has the advantages that it has high affinity to antigen and can be mass-produced, whereas, it also has the disadvantages that when administered into human body, it can induce human anti-mouse antibody response (hereinafter, it referred as “HAMA response”) in human bodies (Shawler et al., J. Immunol., 135, 1530, 1985).

To overcome these disadvantages, humanized antibody has been developed in which high affinity and specificity of mouse monoclonal antibody is maintaining, while HAMA response is minimizing. Humanized antibody is constructed by grafting the complementarity determining regions (hereinafter, it referred as “CDRs”) of mouse monoclonal antibody to the framework regions of a human antibody by genetic engineering technique. Such a humanized antibody was shown to be much less immunogenic in humans (Riechmann et al., Nature, 332, 323, 1988; Nakatani et al., Protein Engineering, 7, 435, 1994). However, it has been reported that the humanized antibody produced by this method also has the disadvantage that the mouse-derived CDRs in the antibody can elicit immune responses when repeatedly administered into human body (Stephens et al., Immunology, 85, 668-674, 1995; Sharkey et al., Cancer Research, 55, 5935s-5945s, 1995). Therefore, it is expected that when the amino acid residues of CDRs derived from mouse antibody in humanized antibodies are substituted for human antibody residues, the HAMA response will be decreased.

There are 3 heavy chain CDR loops—HCDR1, HCDR2 and HCDR3—and 3 light chain CDR loops—LCDR1, LCDR2 and LCDR3—in the variable regions of the antibody molecule. To produce humanized antibody using previous method in the art, all of the 6 CDR loops are grafted. However, since all of the residues in the CDRs do not directly bind to antigen (Padlan et al., FASEB J., 9, 133, 1995), a humanized antibody constructed by grafting the specificity determining residues of mouse monoclonal antibody that are directly involved in antigen binding (hereinafter, it referred as “SDRs”) to the framework regions of a human antibody would induce less HAMA response than previously known humanized antibodies.

On the other hand, Helper T cell is essential in the activation of the early stages in the immune response and MHC (Major Histocompatibility Complex) class II molecule plays an essential role in the selection and activation of the T cells. MHC class II protein binds to peptide having 9 amino acid residues digested from protein antigen and is expressed on the surface of the antigen-presentation cells. The immune responses are initiated when the T cell is activated by complex formation with the T cell receptor (TCR) which recognizes above MHC class II molecule (Germain, R. N. Cell 76, 287-299, 1994). Therefore, humanized antibody without the peptide sequence that may bind to the MHC molecule would not induce the immune response in humans.

Previously, the present inventors provided a humanized antibody that was constructed by CDR-grafting method using the gene of mouse monoclonal antibody H67 against the surface antigen of HBV in Korea patent registration number 163163 (Sep. 3, 1998), and based on this antibody, humanized antibody HZII-H67 with decreased immunogenicity compared with the above-mentioned humanized antibody had been developed and was filed as Korea patent application number 98-32644.

So, to minimize HAMA response by more humanizing above-mentioned humanized antibody HZII-H67, the present inventors developed humanized antibodies which comprise heavy and light chains having amino acid sequences originated from human antibodies at the HCDR1, HCDR2, HCDR3 and LCDR1, LCDR2, LCDR3 of their variable regions, minimizing human anti-mouse antibody (HAMA) response by eliminating peptide sequences from heavy chain of above humanized antibody which bind MHC class II molecules, and completed the present invention by showing that it minimizes the probability of immune response in human body and has good antigen binding capacity, making it a excellent candidate for prevention and treatment of the hepatitis B virus infection.

### SUMMARY OF THE INVENTION

The object of the present invention is to provide humanized antibody against HBV surface antigen S which minimizes the immunogenicity in the human body while having excellent antigen binding capability and method for preparation thereof in order to prevent and treat hepatitis B virus infection effectively.

### BRIEF DESCRIPTION OF THE DRAWINGS

FIG. 1 shows the result which compares nucleotide and amino acid sequences between the heavy chain variable region of humanized antibody HZII-H67 and the humanized heavy chain variable region mutants of the present invention against surface antigen S of HBV.

FIG. 2 shows diagram illustrating the preparing method of humanized heavy chain mutant I34V in which valine is substituted for the 34^{th} isoleucine of mouse HCDR1.

FIG. 3 shows restriction map of expression vector pcDdA-HzSIIIh-I34V which comprises I34V mutant produced by the method of FIG. 2.

FIG. 4 shows the result of ELISA illustrating antigen binding capacity of the humanized antibody against surface antigen S which has the humanized heavy chain mutant of the present invention,

A; HCDR1 mutant,

B; HCDR2 mutant,

C; HCDR3 mutant,

H/K; humanized antibody HZII-H67 composed of heavy and light chain of HZII-H67

FIG. 5 shows the result of ELISA illustrating antigen binding capacity against surface antigen S of the humanized antibody which comprises more than two humanized heavy chain mutations of the present invention.

FIG. 6 shows the result comparing nucleotide and amino acid sequences between the light chain variable region of humanized antibody HZII-H67 and the humanized light chain variable region mutants of the present invention against surface antigen S of HBV.

FIG. 7 shows diagram illustrating the preparing method of the humanized heavy chain mutant Q89L in which leucine is substituted for the 89^{th} glutamine of mouse LCDR1.

FIG. 8 shows restriction map of expression vector pcDdA-HzSIIIh-Q89L which comprises Q89L mutant produced by method in FIG. 7.

FIG. 9 shows the result of ELISA illustrating antigen binding capacity of the humanized antibody against surface antigen S which has the humanized light chain mutant of the present invention,

H/K; humanized antibody HZII-H67 composed of heavy and light chain of HZII-H67

FIG. 10 shows the result of Western blot analysis illustrating the presence of humanized antibody HzS-III having humanized recombinant heavy chain mutant and light chain mutant of the present invention,

A; HzS-III, B; HZII-H67

FIG. 11 shows the result of ELISA illustrating antigen binding capacity of the humanized antibody HzS-III against HBV surface antigen S which has the humanized recombinant heavy chain mutant and light chain mutant of the present invention.

FIG. 12 shows the result of competitive ELISA illustrating antigen binding affinity of the humanized antibody HzS-III and HzS-IV against HBV surface antigen S which has the humanized recombinant heavy chain mutant and light chain mutant of the present invention.

FIG. 13 shows the result comparing amino acid sequences between heavy chain variable region of humanized antibody HzS-III and HzS-IV of the present invention and wild type mouse monoclonal antibody H67 and humanized antibody HZII-H67.

FIG. 14 shows the result comparing amino acid sequences between light chain variable region of humanized antibody HzS-III of the present invention and wild type mouse monoclonal antibody H67 and humanized antibody HZII-H67.

### DETAILED DESCRIPTION OF THE INVENTION

The present invention provides humanized antibody against HBV surface antigen S comprising heavy and light chains in which amino acid residues of human origin is substituted for amino acid residues of CDRs at heavy and light chain variable region of mouse origin; expression vectors comprising each of the heavy and light chain genes of the humanized antibody; and transformant transfected with above expression vector.

Hereinafter, the present invention is described in more detail.

The present invention provides humanized heavy chain mutant in which amino acid residues at the heavy chain antibody of human origin is substituted for amino acid residues of mouse origin at the HCDR1, HCDR2, HCDR3 region of the corresponding heavy chain variable region of the humanized antibody against HBV surface antigen S, and expression vector comprising the same.

After comparing amino acid sequence at HCDR1, HCDR2 and HCDR3 in the heavy chain variable region of mouse-originated humanized antibody HZII-H67 antibody with corresponding HCDR1, HCDR2 and HCDR3 region of human-originated antibody, the most similar CDR sequence is selected. Then, humanized antibody of the present invention is constructed by substituting amino acid residues from human antibody origin for amino acid residues of mouse origin that do not influence the antigen binding activity.

To substitute amino acid of human origin for mouse-originated amino acid residue at HCDR1 region of the humanized antibody HZII-H67, Asp-Tyr-Asn-Ile-Gln represented by SEQ. ID. NO: 1, the present inventors compared the amino sequence of mouse HCDR1 region with that of the human antibody. In results, it was found that mouse HCDR1 had high homology with HCDR1 amino acid residue Asp-Tyr-Asn-Val-Asn of human HCDR1 antibody sequence represented by SEQ. ID. NO: 2 from the Kabat Database ID Number 35920. So, the present inventors construct HCDR1 mutant where 4^{th} and 5^{th} amino acid residues showing differences were substituted.

To do this, mutant I34V or Q35N is constructed in which valine or asparagine was substituted for isoleucine or glutamine at the 34^{th} or 35^{th} amino acid residue, which shows differences between mouse and human HCDR1 region of the antibodies.

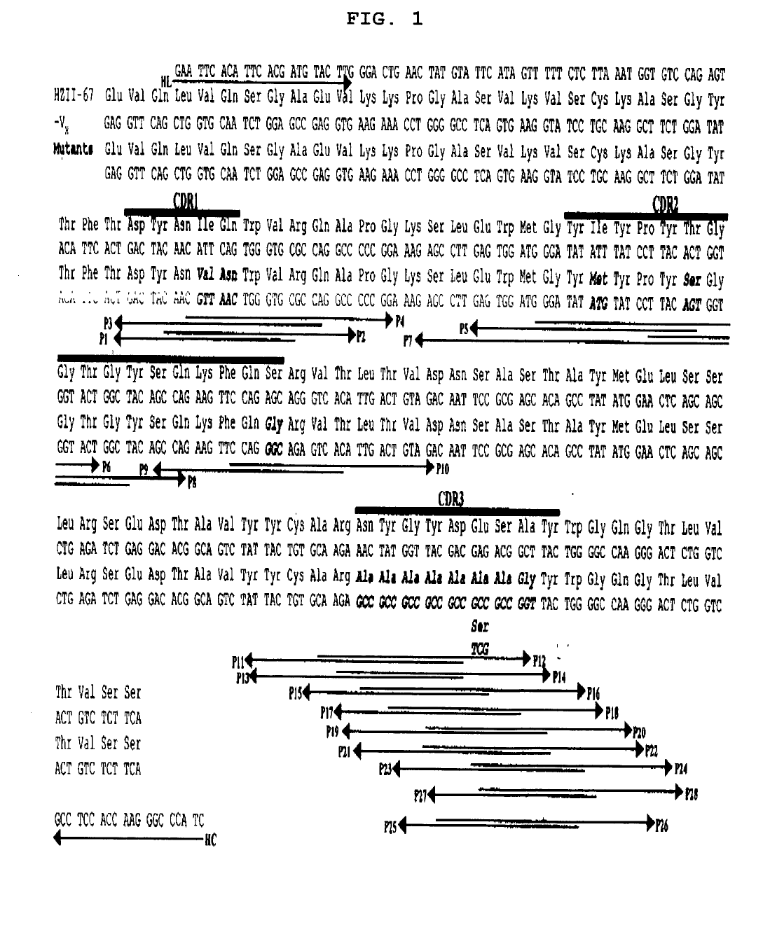

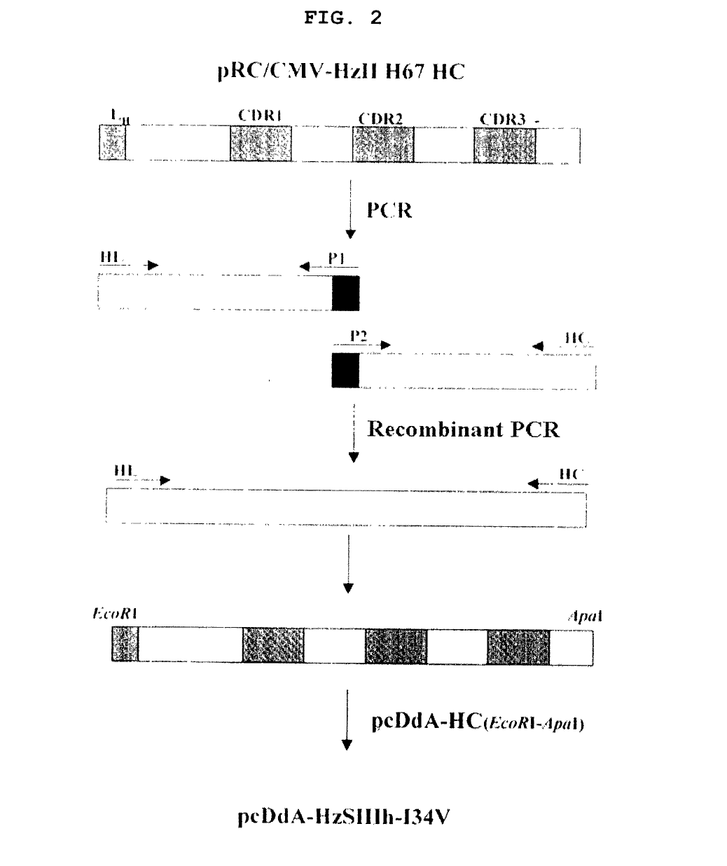

Said mutants are constructed by PCR and recombinant PCR using heavy chain expression vector pRc/CMV-HC-HZIIS (KCTC 0489BP) of humanized antibody as a template (see FIG. 1 and FIG. 2).

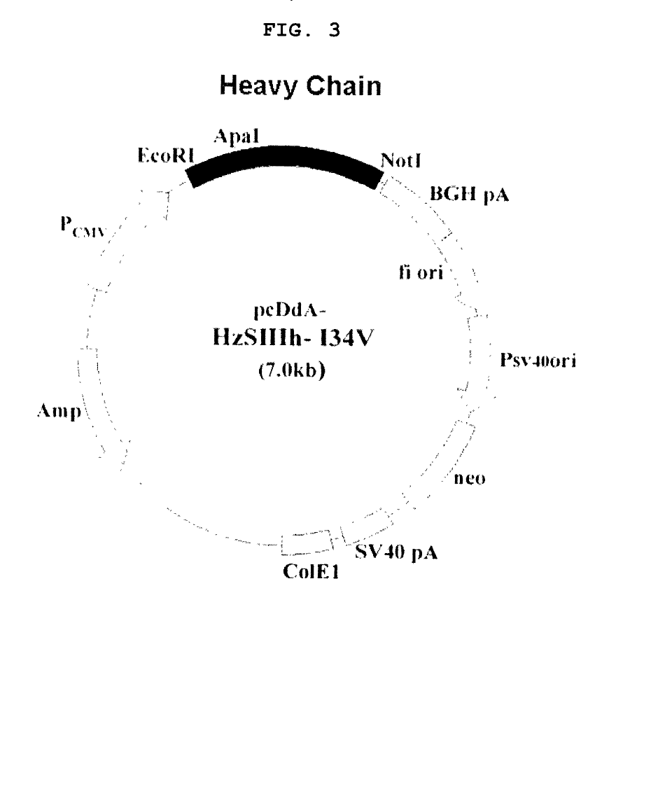

In results, heavy chain variable region mutant I34V of humanized antibody is constructed by performing recombinant PCR reaction using DNA fragments as templates obtained from HL and P1 primer pairs represented by SEQ. ID. No: 3 and SEQ. ID. No: 4 and P2 and HC primer pairs represented by SEQ. ID. No: 5 and SEQ. ID. No: 6, respectively, and HL and HC as primer pairs. After that, recombinant expression vector is constructed by cloning above I34V mutant into expression vector in which cDNA for humanized heavy chain antibody is cloned and was referred as pcDdA-HzSIIIh-I34V (see FIG. 3). After nucleotide sequence analysis of heavy chain variable region I34V mutant of humanized antibody, the present inventors found that heavy chain variable region I34V mutant of humanized antibody was rightly inserted in the above recombinant expression vector.

Q35N mutant is constructed by performing PCR and recombinant PCR by the same method using primer pairs represented by SEQ. ID. No: 3, SEQ. ID. No: 7, SEQ. ID. No: 8 and SEQ. ID. No. 6, in which, among amino acid residues showing differences between HCDR1 sequences at the mouse and human antibodies, asparagine from human heavy chain is substituted for glutamine at the 5^{th} amino acid residue. After that, recombinant expression vector pcDdA-HzSIIIh-Q35N is generated by cloning Q35N into expression vector.

Also, in the humanized antibody of the present invention wherein amino acid residues from human antibody are substituted for amino acid residues at the mouse HCDR2 region of humanized antibody HZII-H67, it is preferred that human-originated HCDR2 amino acid sequence is substituted for all of the mouse-originated HCDR2 amino acid sequence, except amino acid residues directly involved in the antigen binding. To change mouse HCDR2-originated amino acid residues in the humanized antibody HZII-H67 to the human-originated amino acid residues, amino acid sequences at the HCDR2 region of mouse and human origin are compared. After finding the homology between mouse HCDR2 and HCDR2 region at the heavy chain of human antibody DP-15, HCDR2 mutant is constructed using the same method as above by substituting amino acid residues which are not directly involved in the binding to the antigen.

In results, 151M mutant in which methionine is substituted for isoleucine at the 51^{st} amino acid residue of the HCDR2 (using primer pairs represented by SEQ. ID. No: 9 and SEQ. ID. No: 10) and recombinant expression vector pcDdA-HzSIIIh-151M comprising the same, and T54S mutant in which serine is substituted for threonine at the 54^{th} amino acid residue (using primer pairs represented by SEQ. ID. No: 11 and SEQ. ID. No: 12) and recombinant expression vector pcDdA-HzSIIIh-T54S comprising the same, and S65G mutant in which glycine is substituted for serine at the 65^{th} amino acid residue (using primer pairs represented by SEQ. ID. No: 13 and SEQ. ID. No: 14) and recombinant expression vector pcDdA-HzSIIIh-S65G comprising the same are constructed.

In addition, in the humanized antibody of the present invention wherein amino acid residues from human antibody are substituted for amino acid residues at the mouse HCDR3 region of humanized antibody HZII-H67, it is preferred that human-originated HCDR3 amino acid sequence is substituted for all of the mouse-originated HCDR3 amino acid sequence, except amino acid residues directly involved in the antigen binding. To change mouse HCDR3-originated amino acid residues in the humanized antibody HZII-H67 to the human-originated amino acid residues, to begin with, alanine scanning mutagenesis of the mouse HCDR3 was carried out to identify which amino acid residue is directly involved in the antigen binding.

In results, N95A mutant in which alanine is substituted for aspargine at the 95^{th} amino acid residue of the HCDR3 (using primer pairs represented by SEQ. ID. No: 15 and SEQ. ID. No: 16) and recombinant expression vector pcDdA-HzSIIIh-N95A comprising the same, Y96A mutant in which alanine is substituted for tyrosine at the 96^{th} amino acid residue (using primer pairs represented by SEQ. ID. No: 17 and SEQ. ID. No: 18) and recombinant expression vector pcDdA-HzSIIIh-Y96A comprising the same, G97A mutant in which alanine is substituted for glycine at the 97^{th} amino acid residue (using primer pairs represented by SEQ. ID. No: 19 and SEQ. ID. No: 20) and recombinant expression vector pcDdA-HzSIIIh-G97A comprising the same, Y98A mutant in which alanine is substituted for tyrosine at the 98^{th} amino acid residue (using primer pairs represented by SEQ. ID. No: 21 and SEQ. ID. No: 22) and recombinant expression vector pcDdA-HzSIIIh-Y98A comprising the same, D99A mutant in which alanine is substituted for aspargine at the 99^{th} amino acid residue (using primer pairs represented by SEQ. ID. No: 23 and SEQ. ID. No: 24) and recombinant expression vector pcDdA-HzSIIIh-D99A comprising the same, E100A mutant in which alanine is substituted for glutamate at the 100^{th} amino acid residue (using primer pairs represented by SEQ. ID. No: 25 and SEQ. ID. No: 26) and recombinant expression vector pcDdA-HzSIIIh-E100A comprising the same, and S100aA mutant in which alanine is substituted for serine at the 100a^{th} amino acid residue (using primer pairs represented by SEQ. ID. No: 27 and SEQ. ID. No: 28) and recombinant expression vector pcDdA-HzSIIIh-S100aA comprising the same are constructed.

As described hereinbefore, humanized antibody of the present invention is more humanized than that of the previous art by substituting human-originated amino acids for the mouse-originated amino acids, which are not directly involved in the antigen binding and thus are not expected to affect antigen binding affinity, at the CDR region of the humanized antibody. Especially, SDR-grafting method is employed in the present invention to make humanized antibody having less HAMA response and improved therapeutic effect, wherein SDR regions directly involved in the binding to the HBV surface antigen S are substituted into human antibody. That is different from previous methods in which all of the amino acids in the CDR regions of the mouse antibody showing homology are substituted into the human antibody.

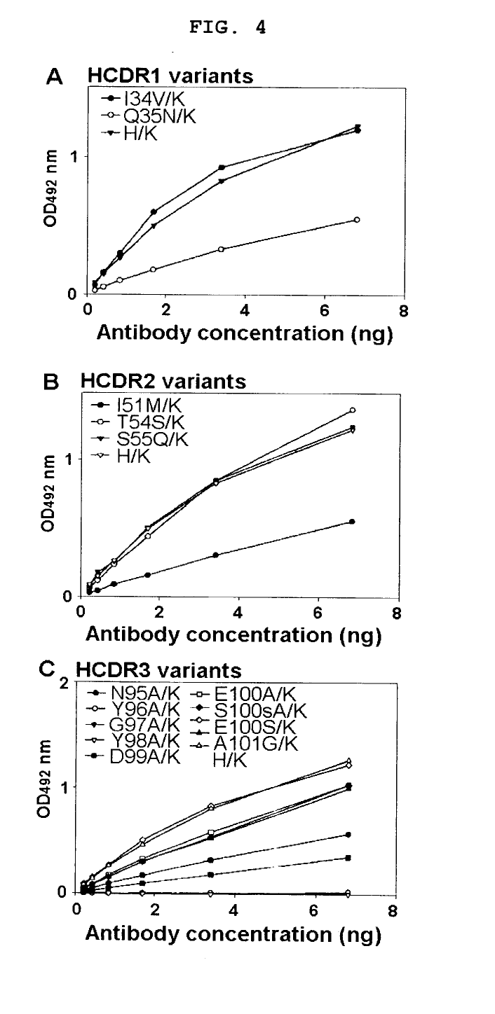

To investigate the expression of humanized antibody of the present invention having mutations at the heavy chain of humanized antibody, the present inventors transfected animal cells with each of the recombinant expression vectors comprising each of the mutant genes at the heavy chain. After measuring the concentrations of antibody that is expressed from above transfectant by sandwich ELISA, antigen binding capacity of the humanized antibody comprising humanized heavy chain mutant against surface antigen S was measured using indirect ELISA. In results, among the HCDR1 mutants, the binding ability of I34V against the HBV surface antigen S was almost the same as that of the heavy chain of the wild type humanized antibody HZS-H67. In the case of HCDR2 mutants, T54S and S65G showed almost the same antigen binding capacity as that of the wild type antibody (see FIG. 4). In the case of HCDR3 mutants, E100A showed slightly lower antigen binding capacity than that of the wild type antibody, indicating that glutamate residue (E100) of the HCDR3 mutant was not important in binding with HBV surface antigen S.

So, the amino acid sequence of human antibody HCDR3 in the data base was compared with that of the mouse HCDR3 in the humanized antibody HZII-H67 in order to substitute the above glutamate residue for that of human antibody. In results, it was found that among human antibody sequences showing high homology with mouse HCDR3 sequence (Kabat Data Base ID No 24562, 39669, 44760), serine and glycine of the 100^{th} and 101^{st} amino acid residue of HCDR3 were consensus amino acid residue. So, the present inventors constructed mutants wherein serine or glycine was substitute for glutamate or alanine of the 100^{th} and 101^{st} amino acid, respectively, and measured the antigen binding capacity of them.

Using the same methods described hereinbefore, E100S mutant in which serine is substituted for glutamate at the 100^{th} amino acid residue (using primer pairs represented by SEQ. ID. No: 29 and SEQ. ID. No: 30) and recombinant expression vector pcDdA-HzSIIIh-E100S comprising the same, and A101G mutant in which glycine is substituted for alanine at the 101^{st} amino acid residue (using primer pairs represented by SEQ. ID. No: 31 and SEQ. ID. No: 32) and recombinant expression vector pcDdA-HzSIIIh-A101G comprising the same are constructed. After measuring antigen binding ability of humanized antibody having humanized heavy chain mutant against surface antigen S, it was found that antigen binding capacity of E100S was similar to that of the wild type, whereas A101G showed slightly decreased antigen binding capacity.

From above results, the present inventors confirmed that among the humanized heavy chain mutant, antigen binding capacity of I34V, T54S, S65G and E100S was similar to that of the wild type. So, we constructed recombinant mutant comprising all of the 4 mutations at the same time.

First, to construct recombinant mutant comprising T54S and I34V mutations, PCR and subcloning were performed using pcDdA-HzSIIIh-T54S as template by the same method described before, and the expression vector constructed from this was referred to as pcDdA-HzSIIIh-T54S+I34V.

Also, to construct recombinant mutant comprising T54S, I34V and E100S mutations, PCR and subcloning were performed using pcDdA-HzSIIIh-I34V as template by the same method described before, and the expression vector obtained from this was referred to as pcDdA-HzSIIIh-T54S+I34V+E100S.

In addition, to construct a recombinant mutant comprising T54S, I34V, E100S and S65G mutations, PCR and subcloning were performed using pcDdA-HzSIIIh-E100s as template by the same method described before. From this, recombinant expression vector pcDdA-HzSIIIh-T54S+I34V+E100S+S65G comprising all of T54S, I34V, E100S and S65G mutations was constructed and referred to as pcDdA-HzSIIIh.

To investigate antigen binding capacity of humanized antibody of the present invention having mutations at the heavy chain of humanized antibody, the present inventors transfected animal cells with the recombinant expression vectors comprising the same and measured antigen binding capacity of the humanized antibody against surface antigen S using indirect ELISA.

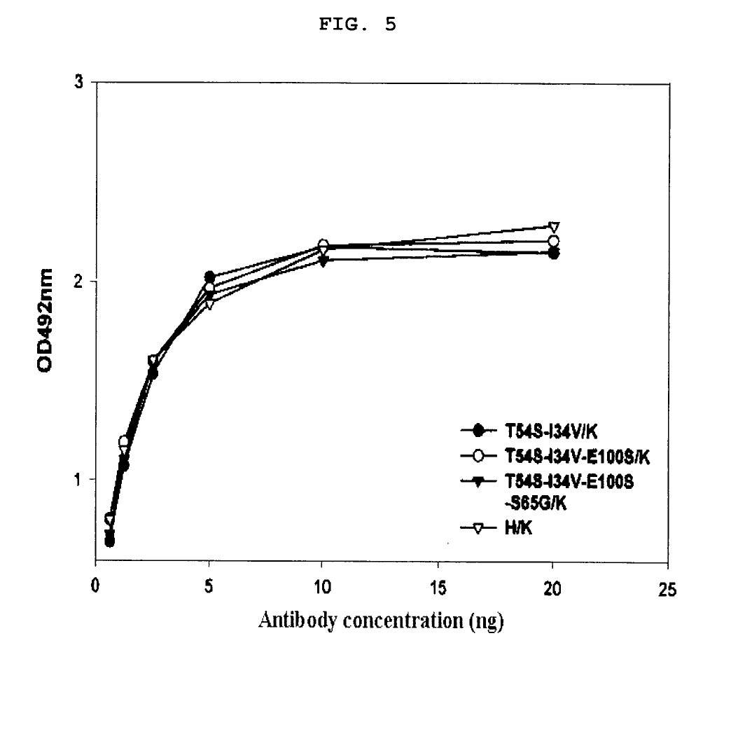

In results, each humanized heavy chain containing mutations of I34V+T54S, I34V+T54S+E100S and I34V+T54S+E100S+S65G, respectively, showed almost the same binding ability against the surface antigen S as that of the wild type humanized antibody HZS-H67 (see FIG. 5). The expression vector pcDdA-HzSIIIh which expresses heavy chain recombinant mutant showing antigen binding capacity against HBV surface antigen S has been deposited in the Korean Collection for Type Culture of Korea Research Institute of Bioscience and Biotechnology (KRIBB) on Sep. 22, 2000 (Accession No: KCTC 10083BP). After analysis of the amino acid sequence of humanized antibody HzS-III which was expressed from expression vector pcDdA-HzSIIIh, the present inventors found that it was composed of humanized heavy chain HzS-III-VH comprising the variable region of amino acid sequences represented by SEQ. ID. No: 43.

Furthermore, the present invention provides humanized light chain mutant produced by SDR-grafting method in which amino acid residues at the light chain antibody of human origin is substituted for amino acid residues at the LCDR1, LCDR2, LCDR3 region of the corresponding light chain variable region of the humanized antibody against HBV surface antigen S, and expression vector comprising the same.

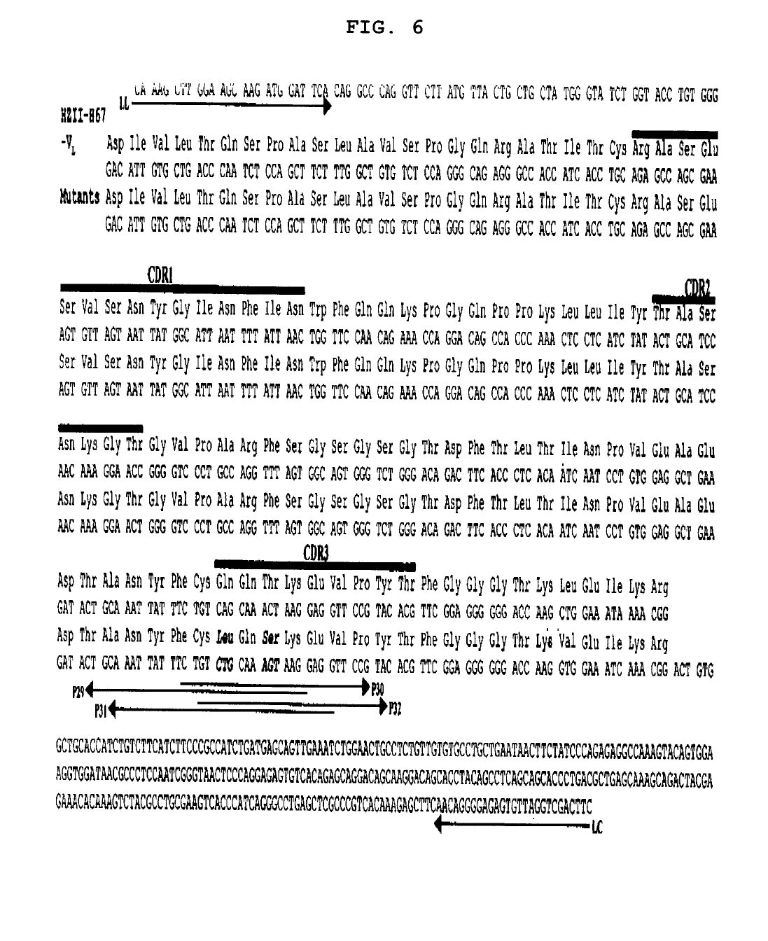

Humanized antibody of the present invention is constructed by substituting human-originated light chain amino acid residues for amino acid residues at the LCDR1, LCDR2 and LCDR3 of the light chain variable region of the humanized antibody HZII-H67. As for the case of humanized light chain, the LCDR1 and LCDR2 of humanized antibody HZII-H67 already has amino acid residues originated from human antibody (in case of LCDR1, D27cS, S31N, M33I; in case of LCDR2, Q54K, S56T). So, the present inventors tried to change the amino acid residues in the LCDR3. In detail, in order to change mouse-originated amino acid residues in LCDR3 region of humanized antibody HZII-H67 to that of human antibody, the present inventors compared the amino acid sequence of humanized light chain with the LCDR3 amino acid sequence of light chain of humanized B1 antibody showing high homology with it (see FIG. 6). From this, the present inventors assumed that the 89^{th} and 91^{st} amino acid residue glutamine and threonine of the mouse-originated LCDR3 are not directly involved in the antigen binding. So, we substituted them with leucine and serine of the amino acid residues in the human antibody.

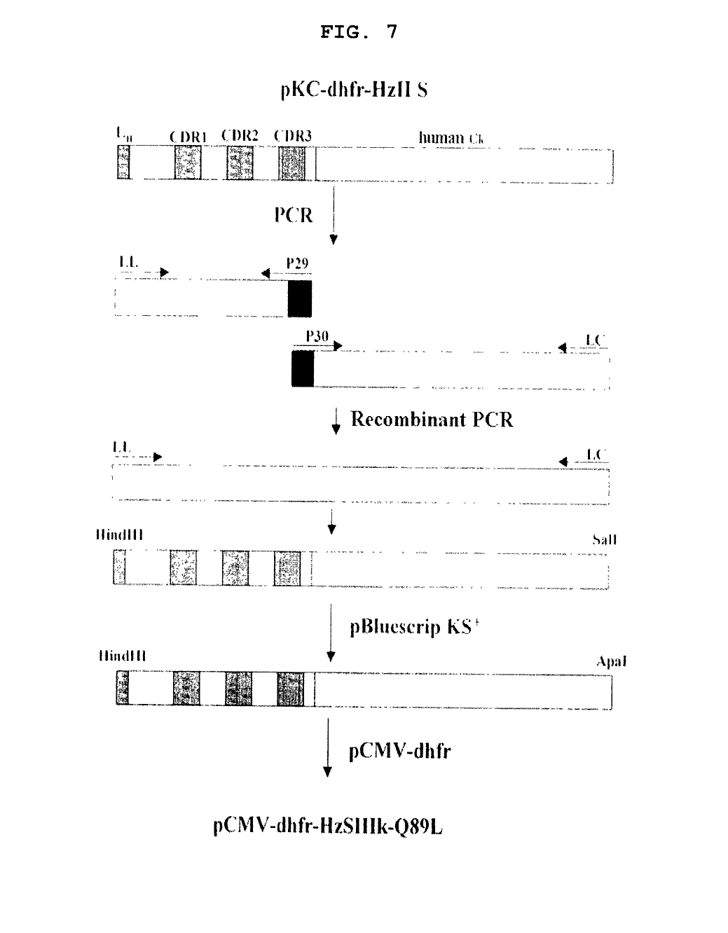

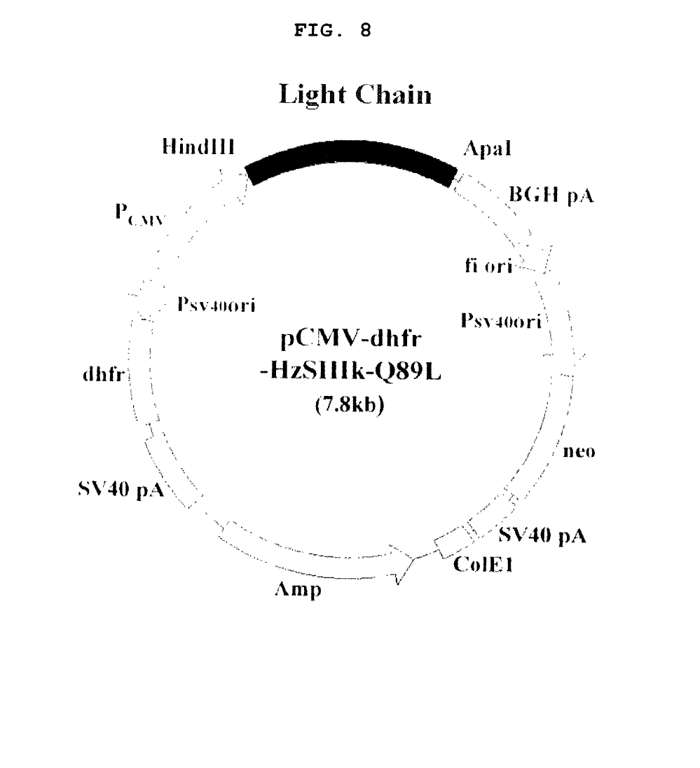

In results, Q89L mutant in which leucine is substituted for glutamine at the 89^{th} amino acid residue (using primer pairs represented by from SEQ. ID. No: 33 to SEQ. ID. No: 36) and recombinant expression vector pcDdA-HzSIIIk-Q89L comprising the same (see FIG. 7 and FIG. 8), and T91S mutant in which serine is substituted for threonine at the 91^{st} amino acid residue (using primer pairs represented by SEQ. ID. No: 33, SEQ. ID. No: 36, SEQ. ID. No: 37 and SEQ. ID. No: 38) and recombinant expression vector pcDdA-HzSIIIk-T91S comprising the same are constructed.

To investigate antigen binding capacity of humanized antibody of the present invention having mutations at the light chain of humanized antibody against HBV surface antigen S, the present inventors transfected animal cells with the recombinant expression vectors comprising each of the mutant and measured antigen binding capacity of the humanized antibody using indirect ELISA.

In results, humanized light chain containing T91S mutant wherein serine was substituted for threonine at the 91^{st} amino acid residue showed the same extent of binding ability against the surface antigen S as that of the heavy chain of the wild type humanized antibody HZS-H67, and expression vector pCMV-dhfr-HzSIIIk-T91S of the present invention expressing T91S mutant was referred as pCMV-dhfr-HzSIIIk. The expression vector pcDdA-HzSIIIk which expresses mutant T91S showing antigen binding capacity against HBV surface antigen S has been deposited in the Korean Collection for Type Culture of Korea Research Institute of Bioscience and Biotechnology (KRIBB) on Sep. 22, 2000 (Accession No: KCTC 10084BP). After analysis of the amino acid sequence of humanized antibody HzS-III expressed from expression vector pcDdA-HzSIIIk, the present inventors found that it was composed of humanized heavy chain HzS-III-VL comprising the variable region of amino acid sequences represented by SEQ. ID. No: 42.

The present invention also provides humanized antibody comprising heavy and light chain gene in which amino acid residues of human origin is substituted for amino acid residues of mouse origin at the heavy and light chain variable regions of the mouse monoclonal antibody H67 against HBV surface antigen S.

To construct humanized antibody comprising recombinant heavy and light chain mutants, the present inventors transfected animal cells with humanized heavy chain expression vector pCDdA-HzSIIIh and humanized light chain expression vector pCMV-dhfr-HzSIIIk. After that, humanized antibody HzS-III having humanized heavy and light chain recombinant mutations at the same time was expressed by incubating selected transformant for 48 hours. After measuring the concentration of humanized antibody HzS-III present in the supernatant of the cultured medium by sandwich ELISA method, antigen binding capacity was measured using indirect ELISA.

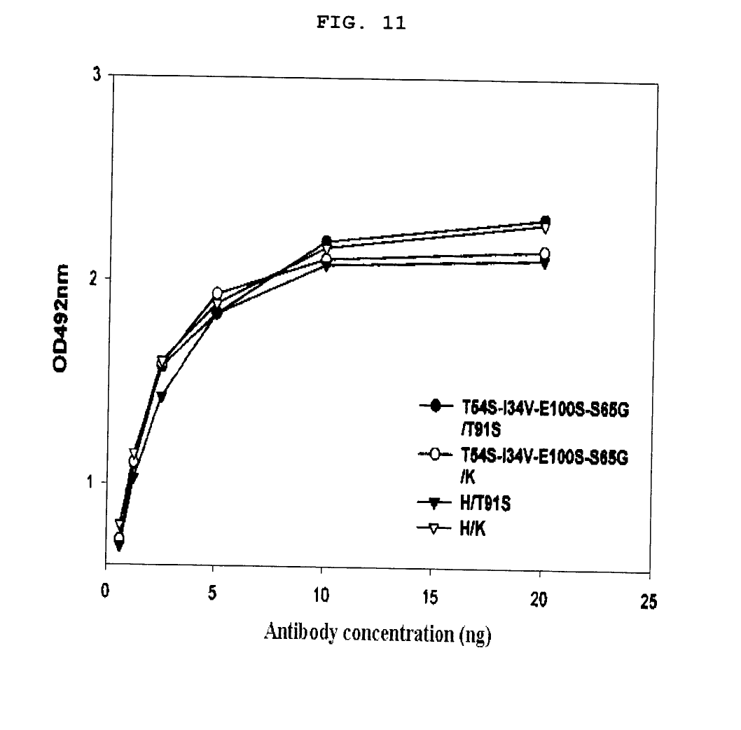

In results, the humanized antibody HzS-III showed binding ability against surface antigen S to almost the same extent as that of the wild type humanized antibody HZS-H67(H/K) (see FIG. 11).

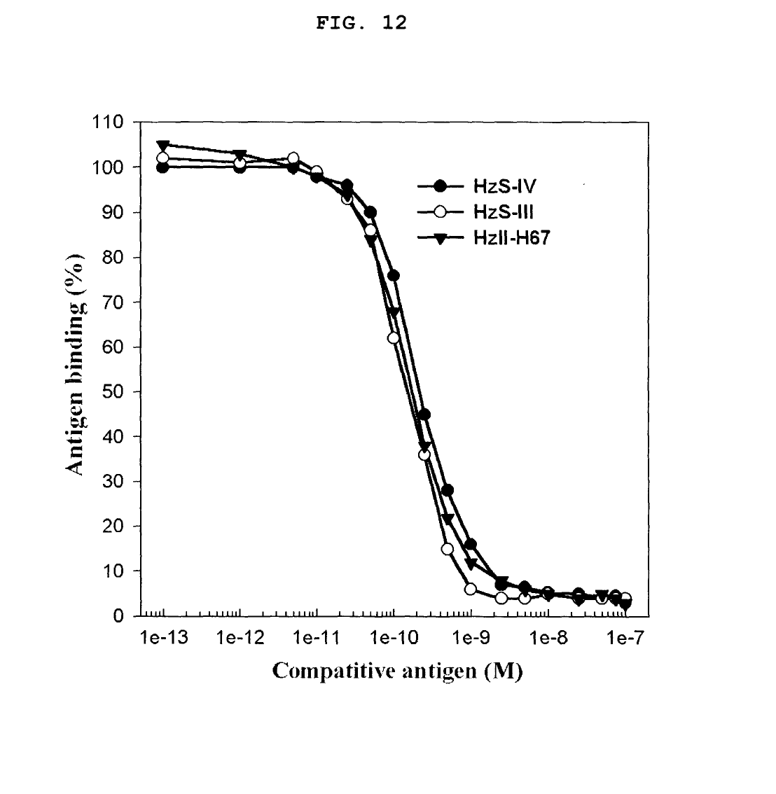

In addition, the present inventors measured antigen binding affinity of humanized antibody HzS-III using competitive ELISA (competitive ELISA, Ryu et al., J. Med. Virol., 52, 226, 1997). In results, it was found that, in spite of the substitution of human-originated amino acid residues for the mouse-originated amino acid residues, antigen binding affinity of humanized antibody HzS-III of the present invention was almost same as that of the wild type humanized antibody HZII-H67 (see FIG. 12).

After screening of peptide sequences which bind to MHC class II molecule in the heavy chain of humanized antibody HzS-III, the present inventors constructed humanized heavy chain substituted by amino acid residues which were not expected to bind to MHC II molecules and measured their antigen binding capacity. In order to produce said humanized heavy chain, S44T mutant in which threonine was substituted for serine at the 44^{th} position (using primer pairs represented by SEQ. ID. No: 46 and 47) and T68D mutant in which and aspartate for threonine at the 68^{th} position (using primer pairs represented by SEQ. ID. No: 48 and 49) and recombinant expression vector pcDdA-HzSIVh containing the same were constructed. The expression vector has been deposited in the Korean Collection for Type Culture of Korea Research Institute of Bioscience and Biotechnology (KRIBB) on Sep. 26, 2001 (Accession No: KCTC 10080BP). After transfecting humanized heavy chain expression vector pcDdA-HzSIVh and humanized light chain expression vector pCMV-dhfr-HzSIIIk into animal cell line, humanized antibody HzS-IV was expressed from above transformant by incubating it for 48 hours. Measuring antigen binding affinity of humanized antibody HzS-IV present in the resulting supernatant, it was found that humanized antibody HzS-IV of the present invention showed almost the same affinity as that of humanized antibody HZS-III (see FIG. 12).

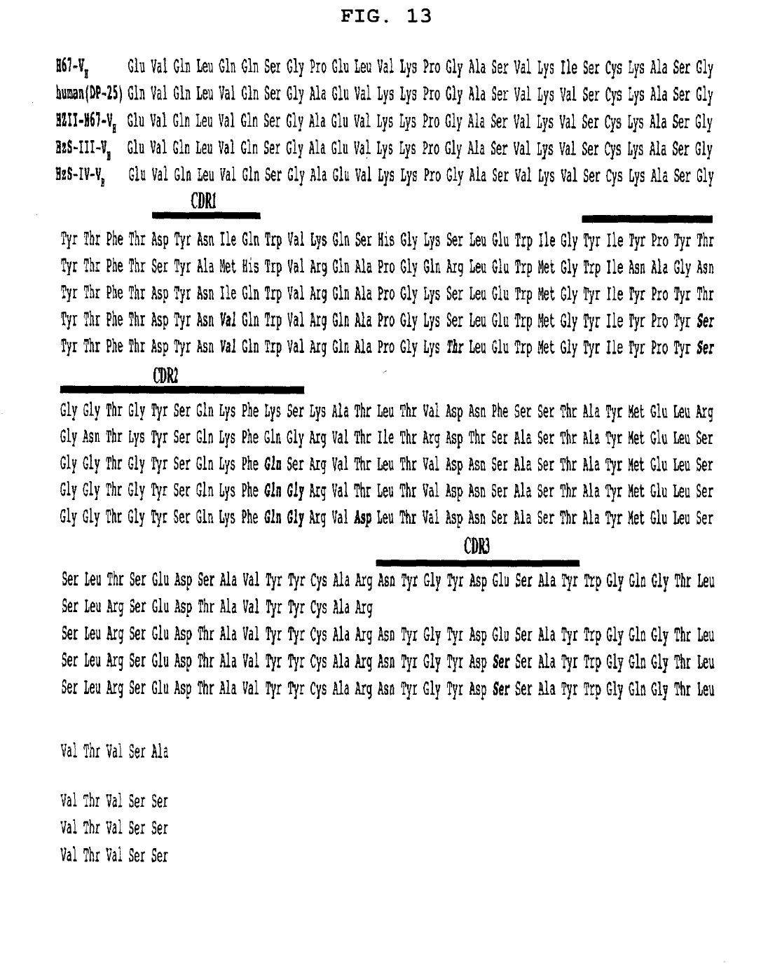

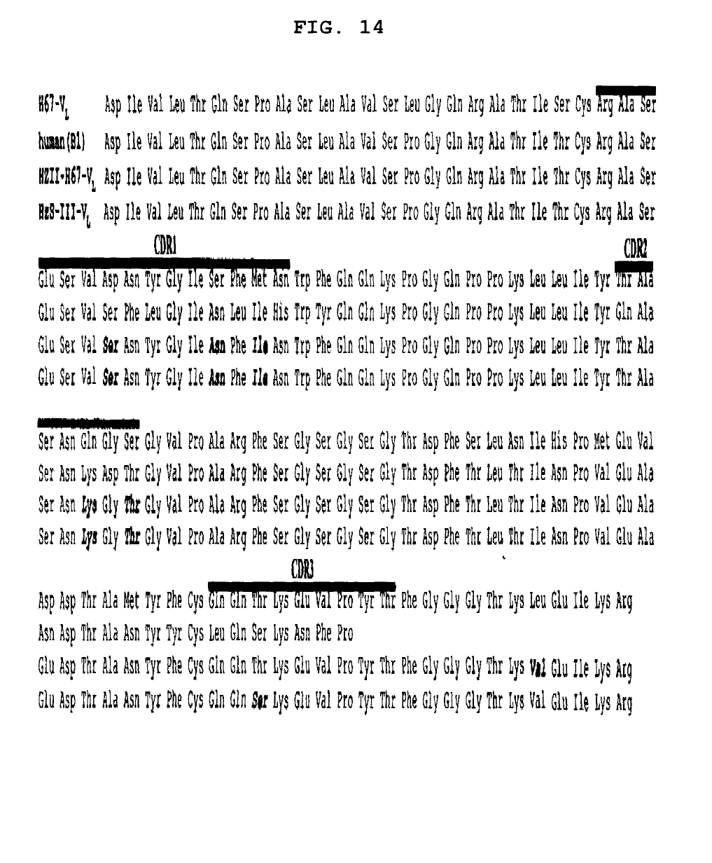

Amino acid sequences of the heavy and light chain variable region of humanized antibody HzS-III and HzS-IV showing excellent binding capacity and antigen binding affinity against HBV surface antigen S was compared with the amino acid sequence of wild type mouse monoclonal antibody H67 and humanized antibody HZII-H67. In results, humanized antibody HzS-III and HzS-IV of the present invention contained less amino acid of mouse origin than that of the mouse monoclonal antibody H67 and humanized antibody HZII-H67, because some of the amino acid residues from mouse origin at HCDR1, HCDR2, HCDR3 of the heavy chain or LCDR1, LCDR2, LCDR3 of the light chain were changed with that of the human origin. Furthermore, among the amino acid sequence at the heavy chain variable region of humanized antibody HzS-IV of the present invention, two amino acid residues at the framework region which was expected to induce immune response by binding to human MHC II molecule were changed, resulting the minimization of the immune responses (see FIG. 13 and FIG. 14).

Therefore, humanized antibody HzS-III and HzS-IV of the present invention can be good candidates for the prevention of hepatitis B virus infection since they show excellent HBV binding activity and induce lower HAMA response than the humanized antibodies of the previous arts.

Hereinafter, we describe the present invention in more detail by Examples.

However, the Examples below are provided to illustrate the present invention and are not included for the purpose of limiting the invention.

### EXAMPLES

### Example 1

### Mutation of the Gene Coding Humanized Heavy Chain Antibody

1-1: Construction of HCDR1 Mutant

To substitute amino acid of human origin for mouse-originated amino acid residue at HCDR1 region of the humanized antibody HZII-H67, Asp-Tyr-Asn-Ile-Gln represented by SEQ. ID. NO: 1 (Korea patent application number 98-32644), the present inventors compared the amino sequence of mouse HCDR1 region with that of the human antibody. In results, it was found that mouse CDR1 had high homology with HCDR1 amino acid residue Asp-Tyr-Asn-Val-Asn of human HCDR1 antibody sequence represented by SEQ. ID. NO: 2 from the Kabat Database ID Number 35920. So, the present inventors constructed HCDR1 mutant where 4^{th} and 5^{th} amino acid residues showing differences were substituted.

1-1-1: Construction of Mutant Where Valine was Substituted for Isoleucine at the 34^{th} Amino Acid Residue

To construct mutant where valine was substituted for isoleucine at the 34^{th} amino acid residue, which shows differences between mouse and human HCDR1 region of the antibodies and is not considered to bind directly at the epitope region of the antigen, PCR and recombinant PCR was performed using primers represented by SEQ. ID. No: 3 to SEQ. ID. No: 6, and heavy chain expression vector of humanized antibody HZII-H67 pRc/CMV-HC-HZIIS (KCTC 0489BP) for template (FIG. 1 and FIG. 2). In detail, PCR was performed by 30 cycles of reaction at 94° C. for 30 seconds, 55° C. for 30 seconds and 72° C. for 1 minute using Taq DNA polymerase (Takara Co.).

In results, it was found that DNA fragment of 181 bp in size was amplified from PCR reaction using HL and P1 primers represented by SEQ. ID. No: 3 and SEQ. ID. No: 4, and DNA fragment of 284 bp in size was amplified from PCR reaction using P2 and HC primers represented by SEQ. ID. No: 5 and SEQ. ID. No: 6. After recombinant PCR reaction using above two DNA fragments as templates and HL and HC primer pairs and by ligation of these two fragments, heavy chain variable region mutant I34V of humanized antibody of 448 bp in size was obtained. After digestion of both ends of variable region of I34V mutant using restriction enzyme EcoRI and ApaI, the present inventors constructed recombinant expression vector by cloning above fragment into the EcoRI-ApaI restriction enzyme site of pcDdA-HC expression vector (expression vector in which heavy chain gene of humanized antibody against preS1 antigen of HBV (Korea patent application number 1998-49663) was cloned at the EcoRI-NotI site of pcDNA2 from Invitrogen company) and referred it as pcDdA-HzSIIIh-I34V (FIG. 3). After nucleotide sequence analysis of heavy chain variable region I34V mutant of humanized antibody, the present inventors found that heavy chain variable region I34V mutant of humanized antibody was rightly inserted in the above recombinant expression vector.

1-1-2: Construction of Mutant Where Asparagine was Substituted for Glutamine at the 35^{th} Amino Acid Residue

To construct mutant Q35N where asparagine was substituted for glutamine at the 35^{th} amino acid residue, which shows differences between mouse and human HCDR1 antibodies and is not considered to bind directly at the epitope region of the antigen, PCR was performed under the same conditions as Example <1-1-1>.

In detail, DNA fragment of 187 bp in size was amplified using HL and P3 primers represented by SEQ. ID. No: 3 and SEQ. ID. No: 7, and DNA fragment of 278 bp in size was amplified using HC and P4 primers represented by SEQ. ID. No: 6 and SEQ. ID. No: 8. Heavy chain variable region mutant of humanized antibody of 448 bp in size, Q35N, was obtained by ligating these two fragments. After digestion of both ends of variable region of Q35N mutant using restriction enzyme EcoRI and ApaI, the present inventors constructed recombinant expression vector by cloning above fragment into the EcoRI-ApaI restriction enzyme site of pcDdA-HC expression vector and referred it as pcDdA-HzSIIIh-Q35N. After nucleotide sequence analysis of heavy chain variable region Q35N mutant of humanized antibody, the present inventors found that heavy chain variable region Q35N mutant of humanized antibody was rightly inserted in the above recombinant expression vector.

1-2: Construction of HCDR2 Mutant

To substitute humanized amino acid for mouse-originated amino acid residue at HCDR2 region of the humanized antibody HZII-H67 represented by SEQ. ID. NO: 39, the present inventors compared the amino sequence of mouse HCDR1 antibody with that of the human origin. In results, it was found that mouse CDR2 had high homology with amino acid sequence of HCDR2 region at the fragment of germline human antibody's heavy chain, DP-15, represented by SEQ. ID. NO: 40. So, the present inventors constructed HCDR2 mutant by substituting amino acid residues which seem not to be involved directly in the binding of antigen.

1-2-1: Construction of Mutant Where Methionine was Substituted for Isoleucine at the 51^{th} Amino Acid Residue

To construct HCDR2 mutant, PCR was performed using P5 and P6 primers represented by SEQ. ID. No: 9 and SEQ. ID. No: 10 instead of P1 and P2 primers by the same method as in Example <1-1-1>. DNA fragment of 236 bp in size was amplified using HL and PS primers, and DNA fragment of 230 bp in size was amplified using HC and P4 primers. After recombinant PCR reaction using above two DNA fragments as templates and HL and HC primer pairs and by ligation of these two fragments, heavy chain variable region mutant 151M of humanized antibody of 448 bp in size was obtained. After digestion of both ends of variable region of the above mutant 151M using restriction enzyme EcoRI and ApaI, the present inventors constructed recombinant expression vector by cloning above fragment into the EcoRI-ApaI restriction enzyme site of pcDdA-HC expression vector and referred it as pcDdA-HzSIIIh-151M. After nucleotide sequence analysis of 151M mutant of heavy chain variable region of humanized antibody, the present inventors found that 151M mutant of heavy chain variable region of humanized antibody was rightly inserted in the above recombinant expression vector.

1-2-2: Construction of Mutant Where Serine was Substituted for Threonine at the 54^{th} Amino Acid Residue

PCR was performed using P7 and P8 primers represented by SEQ. ID. No: 11 and SEQ. ID. No: 12 instead of P1 and P2 primers by the same method as in Example <1-1-1>. DNA fragment of 249 bp in size was amplified using HL and P7 primers, and DNA fragment of 218 bp in size was amplified using HC and P8 primers. After recombinant PCR reaction using above two DNA fragments as templates and HL and HC primer pairs and by ligation of these two fragments, heavy chain variable region mutant T54S of humanized antibody of 448 bp in size was obtained. After digestion of both ends of variable region of the above mutant T54S using restriction enzyme EcoRI and ApaI, the present inventors constructed recombinant expression vector by cloning above fragment into the EcoRI-ApaI restriction enzyme site of pcDdA-HC expression vector and referred it as pcDdA-HzSIIIh-T54S. After nucleotide sequence analysis of T54S mutant of heavy chain variable region of humanized antibody, the present inventors found that T54S mutant of heavy chain variable region of humanized antibody was rightly inserted in the above recombinant expression vector.

1-2-3: Construction of Mutant Where Serine was Substituted for Glycine at the 65^{th} Amino Acid Residue

PCR was performed using P9 and P10 primers represented by SEQ. ID. No: 13 and SEQ. ID. No: 14 instead of P1 and P2 primers by the same method as in Example <1-1-1>. DNA fragment of 278 bp in size was amplified using HL and P9 primers, and DNA fragment of 185 bp in size was amplified using HC and Plo primers. After recombinant PCR reaction using above two DNA fragments as templates and HL and HC primer pairs and by ligation of these two fragments, heavy chain variable region mutant S65G of humanized antibody of 448 bp in size was obtained. After digestion of both ends of variable region of the above mutant S65G using restriction enzyme EcoRI and ApaI, the present inventors constructed recombinant expression vector by cloning above fragment into the EcoRI-ApaI restriction enzyme site of pcDdA-HC expression vector and referred it as pcDdA-HzSIIIh-S65G. After nucleotide sequence analysis of S65G mutant of heavy chain variable region of humanized antibody, the present inventors found that S65G mutant of heavy chain variable region of humanized antibody was rightly inserted in the above recombinant expression vector.

1-3: Construction of HCDR3 Mutant

To substitute humanized amino acid for mouse originated amino acid residue at HCDR3 of the humanized antibody HZII-H67 represented by SEQ. ID. NO: 41, the present inventors compared the amino sequences of mouse HCDR3 antibody with that of the human origin. After that, the present inventors constructed HCDR3 mutant by substituting amino acid residues in the mouse HCDR3 sequences which were seemed not to be involved directly in the binding to the antigen.

1-3-1: Construction of Mutant Where Alanine was Substituted for Aspargine at the 95^{th} Amino Acid Residue

PCR was performed using P11 and P12 primers represented by SEQ. ID. No: 15 and SEQ. ID. No: 16 instead of P1 and P2 primers by the same method as in Example <1-1-1>. DNA fragment of 381 bp in size was amplified using HL and P11 primers, and DNA fragment of 88 bp in size was amplified using HC and P12 primers. After recombinant PCR reaction using above two DNA fragments as templates and HL and HC primer pairs and by ligation of these two fragments, heavy chain variable region mutant N95A of humanized antibody of 448 bp in size was obtained. After digestion of both ends of variable region of the above mutant N95A using restriction enzyme EcoRI and ApaI, the present inventors constructed recombinant expression vector by cloning above fragment into the EcoRI-ApaI restriction enzyme site of pcDdA-HC expression vector and referred it as pcDdA-HzSIIIh-N95A. After nucleotide sequence analysis of N95A mutant of heavy chain variable region of humanized antibody, the present inventors found that N95A mutant of heavy chain variable region of humanized antibody was rightly inserted in the above recombinant expression vector.

1-3-2: Construction of Mutant Where Alanine was Substituted for Tyrosine at the 96^{th} Amino Acid Residue

PCR was performed using P13 and P14 primers represented by SEQ. ID. No: 17 and SEQ. ID. No: 18 instead of P1 and P2 primers by the same method as in Example <1-1-1>. DNA fragment of 380 bp in size was amplified using HL and P13 primers, and DNA fragment of 85 bp in size was amplified using HC and P14 primers. After recombinant PCR reaction using above two DNA fragments as templates and HL and HC primer pairs and by ligation of these two fragments, heavy chain variable region mutant Y96A of humanized antibody of 448 bp in size was obtained. After digestion of both ends of variable region of the above mutant Y96A using restriction enzyme EcoRI and ApaI, the present inventors constructed recombinant expression vector by cloning above fragment into the EcoRI-ApaI restriction enzyme site of pcDdA-HC expression vector and referred it as pcDdA-HzSIIIh-Y96A. After nucleotide sequence analysis of Y96A mutant of heavy chain variable region of humanized antibody, the present inventors found that Y96A mutant of heavy chain variable region of humanized antibody was rightly inserted in the above recombinant expression vector.

1-3-3: Construction of Mutant Where Alanine was Substituted for Glycine at the 97^{th} Amino Acid Residue

PCR was performed using P15 and P16 primers represented by SEQ. ID. No: 19 and SEQ. ID. No: 20 instead of P1 and P2 primers by the same method as in Example <1-1-1>. DNA fragment of 383 bp in size was amplified using HL and P15 primers, and DNA fragment of 82 bp in size was amplified using HC and P14 primers. After recombinant PCR reaction using above two DNA fragments as templates and HL and HC primer pairs and by ligation of these two fragments, heavy chain variable region mutant G97A of humanized antibody of 448 bp in size was obtained. After digestion of both ends of variable region of the above mutant G97A using restriction enzyme EcoRI and ApaI, the present inventors constructed recombinant expression vector by cloning above fragment into the EcoRI-ApaI restriction enzyme site of pcDdA-HC expression vector and referred it as pcDdA-HzSIIIh-G97A. After nucleotide sequence analysis of Y96A mutant of heavy chain variable region of humanized antibody, the present inventors found that G97A mutant of heavy chain variable region of humanized antibody was rightly inserted in the above recombinant expression vector.

1-3-4: Construction of Mutant Where Alanine was Substituted for Tyrosine at the 98^{th} Amino Acid Residue

PCR was performed using P17 and P18 primers represented by SEQ. ID. No: 21 and SEQ. ID. No: 22 instead of P1 and P2 primers by the same method as in Example <1-1-1>. DNA fragment of 386 bp in size was amplified using HL and P17 primers, and DNA fragment of 78 bp in size was amplified using HC and P18 primers. After recombinant PCR reaction using above two DNA fragments as templates and HL and HC primer pairs and by ligation of these two fragments, heavy chain variable region mutant Y98A of humanized antibody of 448 bp in size was obtained. After digestion of both ends of variable region of the above mutant Y98A using restriction enzyme EcoRI and ApaI, the present inventors constructed recombinant expression vector by cloning above fragment into the EcoRI-ApaI restriction enzyme site of pcDdA-HC expression vector and referred it as pcDdA-HzSIIIh-Y98A. After nucleotide sequence analysis of Y98A mutant of heavy chain variable region of humanized antibody, the present inventors found that Y98A mutant of heavy chain variable region of humanized antibody was rightly inserted in the above recombinant expression vector.

1-3-5: Construction of Mutant Where Alanine was Substituted for Aspartate at the 99^{th} Amino Acid Residue

PCR was performed using P19 and P20 primers represented by SEQ. ID. No: 23 and SEQ. ID. No: 24 instead of P1 and P2 primers by the same method as in Example <1-1-1>. DNA fragment of 390 bp in size was amplified using HL and P19 primers, and DNA fragment of 73 bp in size was amplified using HC and P20 primers. After recombinant PCR reaction using above two DNA fragments as templates and HL and HC primer pairs and by ligation of these two fragments, heavy chain variable region mutant D99A of humanized antibody of 448 bp in size was obtained. After digestion of both ends of variable region of the above mutant D99A using restriction enzyme EcoRI and ApaI, the present inventors constructed recombinant expression vector by cloning above fragment into the EcoRI-ApaI restriction enzyme site of pcDdA-HC expression vector and referred it as pcDdA-HzSIIIh-D99A. After nucleotide sequence analysis of Y98A mutant of heavy chain variable region of humanized antibody, the present inventors found that D99A mutant of heavy chain variable region of humanized antibody was rightly inserted in the above recombinant expression vector.

1-3-6: Construction of Mutant Where Alanine was Substituted for Glutamate at the 100^{th} Amino Acid Residue

PCR was performed using P21 and P22 primers represented by SEQ. ID. No: 25 and SEQ. ID. No: 26 instead of P1 and P2 primers by the same method as in Example <1-1-1>. DNA fragment of 390 bp in size was amplified using HL and P21 primers, and DNA fragment of 73 bp in size was amplified using HC and P22 primers. After recombinant PCR reaction using above two DNA fragments as templates and HL and HC primer pairs and by ligation of these two fragments, heavy chain variable region mutant E100A of humanized antibody of 448 bp in size was obtained. After digestion of both ends of variable region of the above mutant E100A using restriction enzyme EcoRI and ApaI, the present inventors constructed recombinant expression vector by cloning above fragment into the EcoRI-ApaI restriction enzyme site of pcDdA-HC expression vector and referred it as pcDdA-HzSIIIh-E100A. After nucleotide sequence analysis of Y98A mutant of heavy chain variable region of humanized antibody, the present inventors found that E100A mutant of heavy chain variable region of humanized antibody was rightly inserted in the above recombinant expression vector.

1-3-7: Construction of Mutant Where Alanine was Substituted for Serine at the 100a^{th} Amino Acid Residue

PCR was performed using P23 and P24 primers represented by SEQ. ID. No: 27 and SEQ. ID. No: 28 instead of P1 and P2 primers by the same method as in Example <1-1-1>. DNA fragment of 395 bp in size was amplified using HL and P23 primers, and DNA fragment of 67 bp in size was amplified using HC and P24 primers. After recombinant PCR reaction using above two DNA fragments as templates and HL and HC primer pairs and by ligation of these two fragments, heavy chain variable region mutant S100aA of humanized antibody of 448 bp in size was obtained. After digestion of both ends of variable region of the above mutant S100aA using restriction enzyme EcoRI and ApaI, the present inventors constructed recombinant expression vector by cloning above fragment into the EcoRI-ApaI restriction enzyme site of pcDdA-HC expression vector and referred it as pcDdA-HzSIIIh-S100aA. After nucleotide sequence analysis of Y98A mutant of heavy chain variable region of humanized antibody, the present inventors found that S100aA mutant of heavy chain variable region of humanized antibody was rightly inserted in the above recombinant expression vector.

### Example 2

### The Expression of Humanized Antibody Having Humanized Heavy Chain Mutant

In order to directly express humanized antibody having humanized heavy chain mutant in the cells, the expression vectors of the present invention were transfected into animal cell lines.

In detail, the COS7 cells were sub-cultured in DMEM medium (GIBCO) supplied with 10% fetal bovine serum at 37° C., 5% CO₂ incubator. The cells were seeded to the 100 mm culturing dish at 1×10⁶ cells/ml concentrations and cultured overnight at 37° C. After that, they were washed 3 times with OPTI-MEM I (GIBCO). Meanwhile, 5 μg of each recombinant expression vector comprising humanized heavy chain mutant of I34V, Q35N, i51M, T54S, S65G, N95A, Y96A, G97A, Y98A, D99A, E100A or S100aA constructed in Example 1 and light chain expression vector pKC-dhfr-HZIIS (KCTC 0490BP) of humanized antibody HZII were diluted with 800 μl of OPTI-MEM I, and 50 μl of Lipofectamine (GIBCO) was also diluted with 800 μl of OPTI-MEM I. DNA-lipofectamine complex was produced by mixing the diluted solutions in 15 ml tube and was incubated at room temperature for at least 15 minutes. 6.4 ml of OPTI-MEM I was added to each of the DNA-Lipofectamine complex and it was transfected by uniformly mixing them with washed COS7 cells. Transfection was performed by incubating cells treated with DNA-Lipofectamine complex at 37° C., 5% CO₂ incubator for 48 hours. Then, supernatant was collected and antibody concentration was measured using sandwich ELISA method. In order to perform sandwich ELISA, anti-human IgG (Sigma Co.) was used as capture antibody and anti-human antibody (Fc specific) combined with horseradish peroxidase (Sigma Co.) was used as secondary antibody.

### Example 3

### Measurement of Antigen Binding Capacity Against the Surface Antigen S of Humanized Antibody Having Humanized Heavy Chain Mutant

The surface antigen S (Greencross Co.) of HBV was coated by adding it to each well of the micro plate in the amount of 250 ng. Each medium transfected with DNA-lipofectamine complex obtained from the above Example 2 was added to the plate so that the concentration of each antibody based on the antibody concentration measured in Example 2 might be 0, 0.2, 0.4, 0.8, 1.8, 3.5 and 7 ng, respectively. The optical density was measured at 492 nm after indirect ELISA which was performed by adding anti-human antibody as a secondary antibody combined with horseradish peroxidase to above well plate. As a control, the same experiment was performed using HZII-H67 pRc/CMV-HC-HZIIS (KCTC 0489BP) and pKC-dhfr-HZIIS as heavy and light chain expression vector, respectively. The result was shown in FIG. 4.

As was indicated in FIG. 4, among the HCDR1 mutants, the binding ability of I34V against the HBV surface antigen S was almost the same as that of the heavy chain of the wild type humanized antibody HZS-H67. In the case of HCDR2 mutants, T54S and S65G showed almost the same extent of antigen binding capacity as that of the wild type antibody. On the other hand, in the case of HCDR3 mutants, E100A showed slightly lower antigen binding capacity than that of the wild type antibody, indicating that glutamate residue (E100) of the HCDR3 mutant was not important in the binding with HBV surface antigen S.

### Example 4

### Construction of HCDR2 Mutant

The present inventors confirmed in the above Example 3 that 100^{th} amino acid residue glutamate (E100) from mouse HCDR3 sequence was not important in the binding with HBV surface antigen S. So, we compared it with the amino acid sequence of human antibody CDR3 in the data base in order to substitute the above amino acid residue for that of another human antibody.

In results, it was found that among human antibody sequences showing high homology with mouse HCDR3 sequence, serine and glycine of the 100^{th} and 101^{st} amino acid residue of HCDR3 were consensus amino acid residue. So, the present inventors constructed mutants wherein serine or glycine was substitute for glutamate or alanine of the 100^{th} and 101^{st} amino acid, respectively, and measured the antigen binding capacity of them.

4-1: Construction of Mutant where Serine was Substituted for Glutamate at the 100^{th} Amino Acid Residue

PCR was performed using P25 and P26 primers represented by SEQ. ID. No: 29 and SEQ. ID. No: 30 instead of P1 and P2 primers by the same method as in Example <1-1-1>. DNA fragment of 390 bp in size was amplified using HL and P25 primers, and DNA fragment of 73 bp in size was amplified using HC and P26 primers. After recombinant PCR reaction using above two DNA fragments as templates and HL and HC primer pairs and by ligation of these two fragments, heavy chain variable region mutant E100A of humanized antibody of 448 bp in size was obtained. After digestion of both ends of variable region of the above mutant E100A using restriction enzyme EcoRI and ApaI, the present inventors constructed recombinant expression vector by cloning above fragment into the EcoRI-ApaI restriction enzyme site of pcDdA-HC expression vector and referred it as pcDdA-HzSIIIh-E100A. After nucleotide sequence analysis of E100A mutant of heavy chain variable region of humanized antibody, the present inventors found that E100A mutant of heavy chain variable region of humanized antibody was rightly inserted in the above recombinant expression vector.

4-2: Construction of Mutant Where Glycine was Substituted for Alanine at the 101^{st} Amino Acid Residue

PCR was performed using P27 and P28 primers represented by SEQ. ID. No: 31 and SEQ. ID. No: 32 instead of P1 and P2 primers by the same method as in Example <1-1-1>. DNA fragment of 396 bp in size was amplified using HL and P27 primers, and DNA fragment of 68 bp in size was amplified using HC and P28 primers. After recombinant PCR reaction using above two DNA fragments as templates and HL and HC primer pairs and by ligation of these two fragments, heavy chain variable region mutant A101G of humanized antibody of 448 bp in size was obtained. After digestion of both ends of variable region of the above mutant A101G using restriction enzyme EcoRI and ApaI, the present inventors constructed recombinant expression vector by cloning above fragment into the EcoRI-ApaI restriction enzyme site of pcDdA-HC expression vector and referred it as pcDdA-HzSIIIh-A101G. After nucleotide sequence analysis of A101G mutant of heavy chain variable region of humanized antibody, the present inventors found that A101G mutant of heavy chain variable region of humanized antibody was rightly inserted in the above recombinant expression vector.

4-3: Measurement of Antigen Binding Capacity Against Surface Antigen S

After transfecting the 2 recombinant expression vector constructed in the above Example <4-1> and <4-2> to the COS cells using the same method as in the Example 2 and 3, the present inventors measured antigen binding capacity of selected transformant using indirect ELISA method. In results, antigen binding capacity of E100S was similar to that of the wild type, whereas A101G showed slightly decreased antigen binding capacity.

From the results of Example 3 and <4-3>, the present inventors confirmed that among the humanized heavy chain mutant, antigen binding capacity of I34V, T54S, S65G and E100S was similar to that of the wild type. So, we constructed recombinant mutant comprising all of the 4 mutations at the same time.

### Example 5

### Recombinant Mutant of Humanized Heavy Chain Gene

5-1: Construction of Recombinant Mutant T54S+I34V

To construct recombinant mutant comprising T54S and I34V mutations, PCR and subcloning were performed using heavy chain gene expression vector pcDdA-HzSIIIh-T54S of the above Example <1-2-1> as templates by the same method as in Example <1-1-1>. The expression vector constructed from this was referred to as pcDdA-HzSIIIh-T54S+I34V.

5-2: Construction of Recombinant Mutant T54S+I34V+E100S

To construct recombinant mutant comprising T54S, I34V and E100S mutations, PCR and subcloning were performed using the heavy chain gene expression vector pcDdA-HzSIIIh-I34V of the above Example <5-1> as templates by the same method as in Example <1-1-1>. The expression vector obtained from this was referred to as pcDdA-HzSIIIh-T54S+I34V+E100S.

5-3: Construction of Recombinant Mutant T54S+I34V+E100S+S65G

To construct a recombinant mutant comprising T54S, I34V, E100S and S65G mutations, PCR and subcloning were performed using heavy chain expression vector pcDdA-HzSIIIh-E100s of the above Example <5-2> as templates by the same method as in Example <1-1-1>. In results, recombinant expression vector pcDdA-HzSIIIh-T54S+I34V+E100S+S65G comprising all of T54S, I34V, E100S and S65G mutations was constructed and referred to as pcDdA-HzSIIIh.

### Example 6

### Measurement of the Antigen Binding Capacity of Humanized Antibody Having Humanized Heavy Chain Recombinant Mutant

After coating the surface antigen S of HBV (Greencross Co.) by adding it to each well of the microplate in the amount of 250 ng, COS7 cell was transfected with each of the recombinant expression vectors or pKC-dhfr-HZIIS vector constructed in the above Example <4-1> to <4-3> by the same method as in Example 2. Each medium containing above transfected cells was added to the plate so that the concentration of each antibody might be 0.625, 1.25, 2.5, 5, 10 and 20 ng, respectively. Then, the optical density was measured using the same method of Example 2. As a control, the same experiment was performed using pRc/CMV-HC-HZIIS and pKC-dhfr-HZIIS as heavy and light chain expression vector of HZII-H67, respectively. The result was shown in FIG. 5.

As was indicated in FIG. 5, each humanized heavy chain containing recombinant mutant of I34V+T54S, I34V+T54S+E100S and I34V+T54S+E100S+S65G, respectively, in which several mutations arose at the same time, showed almost the same binding ability against the surface antigen S as that of the heavy chain of the wild type humanized antibody HZS-H67. The expression vector pcDdA-HzSIIIh which expresses heavy chain recombinant mutant showing antigen binding capacity against HBV surface antigen S has been deposited in the Korean Collection for Type Culture of Korea Research Institute of Bioscience and Biotechnology (KRIBB) on Sep. 22, 2000 (Accession No: KCTC 10083BP). After analysis of the amino acid sequence of humanized antibody HzS-III which was expressed from expression vector pcDdA-HzSIIIh, the present inventors found that it was composed of humanized heavy chain HzS-III-VH comprising the variable region of amino acid sequences represented by SEQ. ID. No: 43.

### Example 7

### Mutation of Humanized Light Chain Gene

As for the case of humanized light chain, the LCDR1 and LCDR2 of humanized antibody HZII-H67 already has amino acid residues originated from human antibody. So, the present inventors tried to change the amino acid residues in the LCDR3. In detail, in order to change mouse-originated amino acid residues in LCDR3 region of humanized antibody HZII-H67 to that of human antibody, the present inventors compared the amino acid sequence of humanized light chain with the LCDR3 amino acid sequence of light chain of humanized B1 antibody showing high homology with it. From this, the present inventors assumed that the 89^{th} and 91^{st} amino acid residue glutamine and threonine of the mouse-originated LCDR3 are not directly involved in the antigen binding. So, we substituted them with leucine and serine of the corresponding light chain residues at the human B1 antibody.

7-1: Construction of Mutant Where Leucine was Substituted for Glutamine at the 89^{th} Amino Acid Residue

To construct mutant where leucine residue of human antibody was substituted for glutamine of the 89^{th} amino acid residue of the mouse-originated LCDR3, PCR and recombinant PCR were performed using primers represented by SEQ. ID. No: 33 and SEQ. ID. No: 36 using light chain expression vector pKC-dhkr-HzIIS of the humanized antibody HZII-H67 as templates (FIG. 6 and FIG. 7).

In detail, 25 cycles of PCR were performed using Taq DNA polymerase at 94° C. for 30 seconds, 55° C. for 30 seconds and 72° C. for 1 minute. DNA fragment of 336 bp in size was amplified from the PCR using LL and P29 primers represented by SEQ. ID. No: 33 and SEQ. ID. No: 36, and DNA fragment of 273 bp in size was amplified from the PCR using P30 and LC primers represented by SEQ. ID. No: 35 and SEQ. ID. No: 36. By performing recombinant PCR reaction using above two DNA fragments as templates and LL and P29 as primer pairs and ligating these two fragments, light chain variable region mutant Q89L of humanized antibody of 743 bp in size was obtained. After digestion of both ends of variable region of the above mutant Q89L using restriction enzyme HindIII and SalI, the present inventors constructed recombinant plasmid pBlue-HzSIIIk-Q89L by cloning at pBluescript KS+ (StrataGen). After nucleotide sequence analysis of G89L mutant of light chain variable region of humanized antibody, the present inventors found that Q89L was rightly inserted in the above expression vector. After obtaining humanized light chain mutant fragment by digesting above pBlue-HzSIIIk-Q89L DNA using restriction enzyme HindIII and ApaI, the present inventors constructed recombinant expression vector by re-cloning above fragment into the HindIII-ApaI site of pCMV-dhfr expression vector (FIG. 7), and referred it as pCMV-dhfr-HzSIIIk-Q89L (FIG. 8).

7-2: Construction of Mutant Where Serine was Substituted for Threonine at the 91^{st} Amino Acid Residue

PCR was performed using primers represented by SEQ. ID. No: 33, SEQ. ID. No: 36, SEQ. ID. No: 37 and SEQ. ID. No: 38 at the same reaction conditions as in Example <7-1>. DNA fragment of 369 bp in size was amplified using LL and P31 primers, and DNA fragment of 391 bp in size was amplified using P32 and LC primers. By performing recombinant PCR reaction using above two DNA fragments as templates and LL and LC primer pairs and ligating these two fragments, light chain variable region mutant T91S of humanized antibody of 743 bp in size was obtained. After digestion of both ends of variable region of the above mutant T91S using restriction enzyme HindIII and SalI, the present inventors constructed recombinant plasmid by cloning at pBluescript KS+. After nucleotide sequence analysis of T91S mutant of light chain variable region of humanized antibody, the present inventors found that T91S was rightly inserted in the above recombinant expression vector.

After obtaining humanized light chain mutant fragment by digesting above pBlue-HzSIIIk-T89S DNA using restriction enzyme HindIII and ApaI, the present inventors constructed recombinant expression vector pCMV-dhfr-HzSIIIk-T91S by re-cloning above fragment into the HindIII-ApaI site of pCMV-dhfr expression vector, and referred it as pCMV-dhfr-HzSIIIk-T91S.

### Example 8

### Measurement of the Binding Ability Against HBV Surface Antigen S of Humanized Antibody Having Humanized Light Chain Mutant

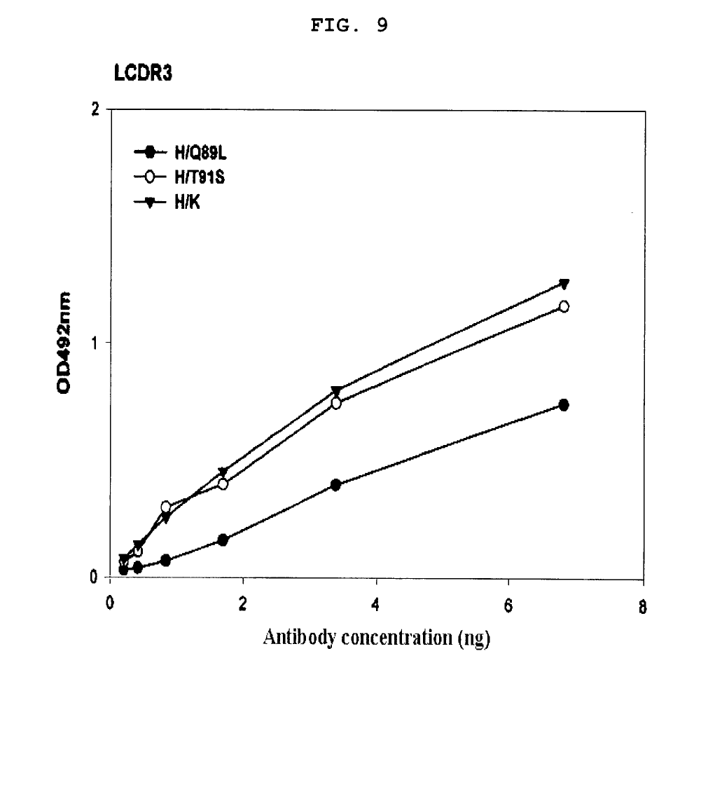

After transfection of COS7 cells with expression vector pCMV-dhfr-HzSIIIk-Q89L or pCMV-dhfr-HzSIIIk-T91S obtained from Example <7-1> and <7-2>, and pRc/CMV-HC-HZIIS vector as heavy chain expression vector of humanized antibody HZII-H67 by the same method of Example 2, selected transformants were incubated for 48 hours. Then, antibody concentration in the supernatant was measured by sandwich ELISA method. Antibody was added to the plate so that the concentration of each antibody might be 0, 0.2, 0.4, 0.8, 1.8, 3.5 and 7 ng, respectively, using the same method as in the Example 3. As a control, the same experiment was performed using pRc/CMV-HC-HZIIS and pKC-dhfr-HZIIS as heavy and light chain expression vector of HZII-H67, respectively. The result was shown in FIG. 9.

As was indicated in FIG. 9, humanized light chain containing T91S mutant wherein serine was substituted for threonine at the 91^{st} amino acid residue showed the same extent of binding ability against the surface antigen S as that of the heavy chain of the wild type humanized antibody HZS-H67. The expression vector pcDdA-HzSIIIk which expresses mutant T91S showing antigen binding capacity against HBV surface antigen S has been deposited in the Korean Collection for Type Culture of Korea Research Institute of Bioscience and Biotechnology (KRIBB) on Sep. 22, 2000 (Accession No: KCTC 10084BP). After analysis of the amino acid sequence of humanized antibody HzS-III expressed from expression vector pcDdA-HzSIIIk, the present inventors found that it was composed of humanized heavy chain HzS-III-VL comprising the variable region of amino acid sequences represented by SEQ. ID. No: 42.

### Example 9

### Measurement of the Antigen Binding Capacity of Humanized Antibody (HzS-III) Having Humanized Heavy and Light Chain Recombinant Mutant

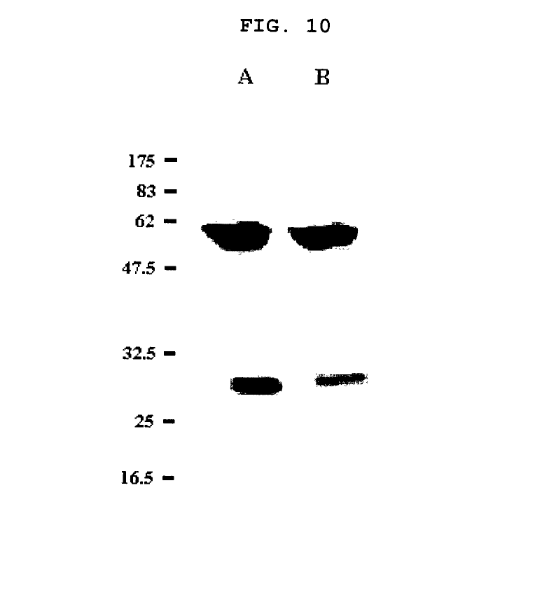

After transfection of COS7 cells with humanized heavy chain expression vector pCDdA-HzSIIIh from Example <5-2> and humanized light chain expression vector pCMV-dhfr-HzSIIIk from Example <7-2> by the same method of Example 2, humanized antibody HzS-III having humanized heavy and light chain recombinant mutations at the same time was expressed by incubating selected transformant for 48 hours. In order to confirm the presence of humanized antibody HzS-III in the supernatant obtained from the cultured medium, Western blot analysis was performed using secondary antibody wherein horseradish peroxidase (Sigma Co.) was combined at the anti-human antibody (Fc specific). In results, as was indicated in FIG. 10, humanized heavy chain of about 55 kDa in size and humanized light chain of about 27 kDa in size were detected. So, the present inventors found that humanized antibody of the present invention was rightly expressed and secreted from the transformant.

After measuring the concentration of secreted antibody in the supernatant by sandwich ELISA method, antibody was added to the plate coated with HBV surface antigen S so that the concentration of each antibody might be 0.625, 1.25, 2.5, 5, 10 and 20 ng, respectively, using the same method as in the Example 3. As a control, the same experiment was performed using pRc/CMV-HC-HZIIS and pKC-dhfr-HZIIS as heavy and light chain expression vector of HZII-H67, respectively. The result was shown in FIG. 11.

As was indicated in FIG. 11, humanized antibody HzS-III having humanized heavy and light chain recombinant mutations at the same time showed binding ability against surface antigen S to almost the same extent as that of the wild type humanized antibody HZS-H67(H/K).

### Example 10

### Determination of Antigen Binding Affinity of Humanized Antibody HzS-III

Competitive ELISA method was used to determine antigen binding affinity of humanized antibody HzS-III (competitive ELISA, Ryu et al., J. Med. Virol., 52, 226, 1997). After pre-incubation of 5 ng of humanized antibody HzS-III produced from the COS7 cell of the Example 8 and surface antigen S (from 1×10⁻⁷ M to 1×10⁻¹² M) at 37° C. for 3 hours, the mixture was added to the 96 well plate pre-coated with surface antigen S. After reaction of the well plate at 37° C. for 3 hours, optical density was measured by ELISA using the same method of Example 3 (FIG. 12). As a control, humanized antibody HZII-H67 was used.

In results, it was found that antigen binding affinity of humanized antibody HzS-III of the present invention was about 4×10⁹ M⁻¹, which did not show large differences with that of the control HZII-H67 (3.2×10⁹ M⁻¹).

### Example 11

### Construction of Humanized Antibody HzS-IV Wherein Peptide Sequence Which Binds to MHC Class II Molecule is Removed from Humanized Antibody HzS-III

The present inventors investigated whether peptide sequence which binds to MHC class II molecule was present at the above humanized antibody HzS-III using Sturniolo's method (TEPITOPE) (Sturniolo et al., Nature Biotechnology, 17, 555-561, 1999). In addition, the present inventors searched sequences from the human germline DP-25 protein showing high homology with that of above humanized antibody, and excluded it from consideration.

In results, it was found that 2 peptide sequences at humanized heavy chain were bound to MHC class II. The first peptide had amino acid sequence represented by SEQ. ID. No: 44 and was found to be bound with 22 MHC class II out of the 51 MHC class II molecules. The second peptide had amino acid sequence represented by SEQ. ID. No: 45 and was found to be bound with 14 MHC class II molecules (Table 1).

TABLE 1
Result of TEPITOPE analysis of humanized
heavy chain HzSIIIh

| | Before | After | Before | After |
|---|---|---|---|---|
| | substitution | substitution | substitution | substitution |
| | WVKQAPGKS | WVKQAPGKT | FQGRVTLTV | FQGRVDLTV |
| Peptide | (SEQ. ID. No.44) | (SEQ. ID. No.46) | (SEQ. ID. No.45) | (SEQ. ID. No.47) |
| MHC class | DRB1_0101 | DRB1_0101 | DRB1_0402 | DRB1*1502 |
| II | DRB1_0301 | DRB1_0305 | DRB1_0421 | |
| molecule | DRB1_0305 | DRB1_0309 | DRB1_0701 | |
| which | DRB1_0309 | DRB1_1101 | DRB1_0703 | |
| binds to | DRB1_0401 | DRB1_1307 | DRB1_0801 | |
| peptide | DRB1_0405 | DRB1_1321 | DRB1_0802 | |
| | DRB1_0408 | DRBS_0101 | DRB1_0804 | |
| | DRB1_0421 | DRB1_0105 | DRB1_0806 | |
| | DRB1_0426 | | DRB1_0813 | |
| | DRB1_0801 | | DRB1_0817 | |
| | DRB1_0802 | | DRB1_1101 | |
| | DRB1_1101 | | DRB1_1114 | |
| | DRB1_1102 | | DRB1_1120 | |
| | DRB1_1104 | | DRB1_1128 | |
| | DBR1_1106 | | DRB1_1302 | |
| | DRB1_1107 | | DRB1_1305 | |
| | DRB1_1114 | | DRB1_1323 | |
| | DRB1_1120 | | DRB1_1502 | |
| | DRB1_1121 | | | |
| | DRB1_1128 | | | |
| | DRB1_1302 | | | |
| | DRB1_1305 | | | |
| | DRB1_1307 | | | |
| | DRB1_1311 | | | |
| | DRB1_1321 | | | |
| | DRB1_1322 | | | |
| | DRB1_1323 | | | |
| | DRB5_0101 | | | |
| | DRB5_0105 | | | |
| Total | 29 | 14 | 18 | 1 |
| number | | | | |

In order to weaken the binding affinity of peptides to MHC II molecules, the present inventors substituted threonine for serine at the P9 position of amino acid sequence represented by SEQ. ID. No: 44, and aspartate for threonine at the P6 position of amino acid sequence represented by SEQ. ID. No: 45. In results, the number of MHC class II molecules which could bind to the peptides was decreased to 11 (55%) in case of SEQ. ID. No: 44. In case of SEQ. ID. No: 45, only 1 molecule among 51 MHC class II molecules could bind to peptide.

Therefore, in order to produce humanized heavy chain with weakened binding affinity against MHC class II molecule, S44T/T68D mutant having mutations of S44T and T68D was constructed, in which threonine was substituted for serine at the 44^{th} position (using primer pairs represented by SEQ. ID. No: 46 and 47) and aspartate for threonine at the 68^{th} position (using primer pairs represented by SEQ. ID. No: 48 and 49), respectively.

After digestion of both ends of variable region of the above mutant S44T/T68D using restriction enzyme EcoRI and ApaI, the present inventors constructed recombinant expression vector by cloning above fragment into the EcoRI-ApaI restriction enzyme site of pcDdA-HC expression vector and referred it as pcDdA-HzSIVh. The expression vector has been deposited in the Korean Collection for Type Culture of Korea Research Institute of Bioscience and Biotechnology (KRIBB) on Sep. 26, 2001 (Accession No: KCTC 10080BP).

After transfecting humanized heavy chain expression vector pcDdA-HzSIVh and humanized light chain expression vector pCMV-dhfr-HzSIIIk into animal cell line, humanized antibody HzS-IV was expressed from above transformant by incubating it for 48 hours. Measuring antigen binding affinity of humanized antibody HzS-IV present in the resulting supernatant, it was found that humanized antibody HzS-IV of the present invention showed almost the same extent of binding affinity as that of humanized antibody HZS-III (see FIG. 12).

### Example 12

### Comparison Between the Amino Acid Sequences of Humanized Antibody HzS-III and HzS-IV

Amino acid sequences of the heavy/light chain variable region of humanized antibody HzS-III and HzS-IV was compared with the amino acid sequence of wild type mouse monoclonal antibody H67 and humanized antibody HZII-H67.

In results, as was indicated in FIG. 13 and FIG. 14, it was expected that humanized antibody HzS-III and HzS-IV of the present invention might cause lower HAMA response, because amino acid sequences of the heavy/light chain variable region of humanized antibody HzS-III and HzS-IV contained less amino acid residues of mouse origin at HCDR1, HCDR2, HCDR3 of the heavy chain or LCDR1, LCDR2, LCDR3 of the light chain than that of the mouse monoclonal antibody H67 and humanized antibody HZII-H67, and HzS-IV had fewer amino acid sequences than HzS-III which could bind to MHC class molecules. Therefore, humanized antibody HzS-III and HzS-IV of the present invention can be good candidates for the prevention of hepatitis B virus since they induce lower HAMA response than the humanized antibodies of the previous arts.

### INDUSTRIAL APPLICABILITY

As described hereinbefore, humanized antibody of the present invention against HBV surface antigen S is more humanized than that of the previous arts by substituting human-originated amino acid residues for mouse-originated amino acid residues at HCDR1, HCDR2, HCDR3 of heavy chain and LCDR1, LCDR2, LCDR3 of light chain. Moreover, it outstandingly decreased the probability of immunogenicity in human body by reducing amino acid sequences in the FR region which could bind to MHC class II molecules and showed excellent antigen binding affinity. Therefore, humanized antibody of the present invention can be useful for the prevention of hepatitis B virus infection.

What is claimed is:

1. Humanized heavy chain mutant which binds specifically to the surface antigen S of hepatitis B virus, wherein all or parts of the amino acid residues in the HCDRs of the heavy chain of human origin are substituted for all or parts of the amino acid residues selected from a group consisting HCDR1, HCDR2 and HCDR3 of the heavy chain of mouse origin.

2. Humanized heavy chain mutant according to claim 1, wherein valine is substituted for isoleucine at the 34^{th} amino acid of HCDR1.

3. Humanized heavy chain mutant according to claim 1, wherein serine is substituted for threonine at the 54^{th} amino acid of HCDR2.

4. Humanized heavy chain mutant according to claim 1, wherein glycine is substituted for serine at the 65^{th} amino acid of HCDR2.

5. Humanized heavy chain mutant according to claim 1, wherein serine is substituted for glutamate at the 100^{th} amino acid of HCDR3.

6. Humanized heavy chain mutant according to claim 1, wherein it contains all of the mutations from claim 2 to claim 5.

7. Expression vector pcDdA-HzSIIIh comprising humanized heavy chain mutant of the claim 6 (Deposition number: KCTC 10083BP).

8. Humanized heavy chain mutant which binds specifically to the surface antigen S of hepatitis B virus, wherein all or parts of the amino acid residues in the HCDRs of the heavy chain of human origin are substituted for all or parts of the amino acid residues selected from a group consisting HCDR1, HCDR2 and HCDR3 of the heavy chain of mouse origin, and peptide sequences binding to MHC class II molecule are removed.

9. Humanized heavy chain mutant according to claim 8, wherein peptide sequences binding to MHC class II molecule is represented by SEQ. ID. No: 44 or SEQ. ID. No: 45.

10. Humanized heavy chain mutant according to claim 8, wherein threonine is substituted for serine at the P9 position of the sequence represented by SEQ. ID. No: 44.

11. Humanized heavy chain mutant according to claim 8, wherein aspartate is substituted for threonene at the P6 position of the sequence represented by SEQ. ID. No: 45.

12. Humanized heavy chain mutant according to claim 8, wherein it contains all of the mutations from claim 10 to claim 11.

13. Expression vector pcDdA-HzSIVh comprising humanized heavy chain mutant of the claim 12 (Deposition number: KCTC 10080BP).

14. Humanized light chain mutant which binds specifically to the surface antigen S of hepatitis B virus, wherein all or parts of the amino acid residues in the LCDRs of the light chain of human origin are substituted for all or parts of the amino acid residues selected from a group consisting LCDR1, LCDR2 and LCDR3 of the light chain of mouse origin.

15. Humanized light chain mutant according to claim 14, wherein serine is substituted for aspartate at the 27c^{th} amino acid of LCDR1.

16. Humanized light chain mutant according to claim 14, wherein aspargine is substituted for serine at the 31^{st} amino acid of LCDR1.

17. Humanized light chain mutant according to claim 14, wherein isoleucine is substituted for methionine at the 33^{rd} amino acid of LCDR1.

18. Humanized light chain mutant according to claim 14, wherein lysine is substituted for glutamine at the 54^{th} amino acid of LCDR2.

19. Humanized light chain mutant according to claim 14, wherein threonine is substituted for serine at the 56^{th} amino acid of LCDR2.

20. Humanized light chain mutant according to claim 14, wherein serine is substituted for threonine at the 91^{st} amino acid of LCDR3.

21. Humanized light chain mutant according to claim 14, wherein it contains all of the mutations from claim 15 to claim 20.

22. Expression vector pCMV-dhfr-HzSIIIk comprising humanized light chain mutant of the claim 21 (Deposition number: KCTC 10084BP).

23. Transformant transfected simultaneously with humanized heavy chain expression vector of claim 7 or claim 13 and humanized light chain expression vector of claim 22.

24. Humanized antibody against surface antigen S of hepatitis B virus, expressed from the transformant of claim 23.

25. Method for the preparation of humanized antibody by comparing the CDR sequences between mouse and human antibody, selecting most homologous human CDR sequences and substituting the amino acid residues in the CDR of human antibody for the amino acid residues in the CDR of mouse antibody, wherein the amino acid residue in the mouse CDR does not affect the antigen binding.


SEQUENCE LISTING (52 sequences)

SEQ ID NO: 1 - protein, 5 aa, Mouse
DYNIQ

SEQ ID NO: 2 - protein, 5 aa, Homo sapiens
DYNVD

SEQ ID NO: 3 - DNA, 26 bp, Artificial Sequence
  HL
gagaattcacattcacgatgtacttg

SEQ ID NO: 4 - DNA, 23 bp, Artificial Sequence
  P1
cccactgaacgttgtagtcagtg

SEQ ID NO: 5 - DNA, 25 bp, Artificial Sequence
  P2
ctacaacgttcagtgggtgcgcaag

SEQ ID NO: 6 - DNA, 20 bp, Artificial Sequence
  HC
gatgggcccttggtggaggc

SEQ ID NO: 7 - DNA, 27 bp, Artificial Sequence
  P3
cgcacccagttaatgttgtagtcagtg

SEQ ID NO: 8 - DNA, 27 bp, Artificial Sequence
  P4
caacattaactgggtgcgccaggcccc

SEQ ID NO: 9 - DNA, 30 bp, Artificial Sequence
  P5
gtaaggatacatatatcccatccactcaag

SEQ ID NO: 10 - DNA, 27 bp, Artificial Sequence
  P6
gggatatatgtatccttacactggtgg

SEQ ID NO: 11 - DNA, 30 bp, Artificial Sequence
  P7
cagtaccaccactgtaaggataaatatatc

SEQ ID NO: 12 - DNA, 26 bp, Artificial Sequence
  P8
ccttacagtggtggtactggctacag

SEQ ID NO: 13 - DNA, 24 bp, Artificial Sequence
  P9
gtgactctgccctggaacttctgg

SEQ ID NO: 14 - DNA, 23 bp, Artificial Sequence
  P10
ccagggcagagtcacattgactg

SEQ ID NO: 15 - DNA, 30 bp, Artificial Sequence
  P11
gtaaccataggctcttgcacagtaatagac

SEQ ID NO: 16 - DNA, 28 bp, Artificial Sequence
  P12
gtgcaagagcctatggttacgacgagtc

SEQ ID NO: 17 - DNA, 28 bp, Artificial Sequence
  P13
gtaaccggcgtttcttgcacagtaatag

SEQ ID NO: 18 - DNA, 27 bp, Artificial Sequence
  P14
caagaaacgccggttacgacgagtctg

SEQ ID NO: 19 - DNA, 25 bp, Artificial Sequence
  P15
gtcgtaggcatagtttcttgcacag

SEQ ID NO: 20 - DNA, 29 bp, Artificial Sequence
  P16
gaaactatgcctacgacgagtctgcctac

SEQ ID NO: 21 - DNA, 26 bp, Artificial Sequence
  P17
ctcgtcggcaccatagtttcttgcac

SEQ ID NO: 22 - DNA, 24 bp, Artificial Sequence
  P18
ctatggtgccgagtctgcttactg

SEQ ID NO: 23 - DNA, 27 bp, Artificial Sequence
  P19
cagactcggcgtaaccatagtttcttg

SEQ ID NO: 24 - DNA, 26 bp, Artificial Sequence
  P20
ggttacggcgagtctgcttactgggg

SEQ ID NO: 25 - DNA, 24 bp, Artificial Sequence
  P21
cagaggcgtcgtaaccatagtttc

SEQ ID NO: 26 - DNA, 27 bp, Artificial Sequence
  P22
ggttacgacgcctctgcttactggggc

SEQ ID NO: 27 - DNA, 25 bp, Artificial Sequence
  P23
gtaagcggcctcgtcgtaaccatgg

SEQ ID NO: 28 - DNA, 26 bp, Artificial Sequence
  P24
gacgaggccgcttactggggccaagg

SEQ ID NO: 29 - DNA, 25 bp, Artificial Sequence
  P25
gtaagcagacgagtcgtaaccatag

SEQ ID NO: 30 - DNA, 26 bp, Artificial Sequence
  P26
cgactcgtctgcttactggggccaag

SEQ ID NO: 31 - DNA, 24 bp, Artificial Sequence
  P27
ccagtaaccagactcgtcgtaacc

SEQ ID NO: 32 - DNA, 25 bp, Artificial Sequence
  P28
cgagtctggttactggggccaaggg

SEQ ID NO: 33 - DNA, 26 bp, Artificial Sequence
  LL
caaagcttggaagcaagatggaatca

SEQ ID NO: 34 - DNA, 30 bp, Artificial Sequence
  P29
ccttagtttgcagacagaaataatttgcag

SEQ ID NO: 35 - DNA, 25 bp, Artificial Sequence
  P30
ctgtctgcaaactaaggcggttccg

SEQ ID NO: 36 - DNA, 27 bp, Artificial Sequence
  LC
gaagtcgacctaacactctcccctgtt

SEQ ID NO: 37 - DNA, 30 bp, Artificial Sequence
  P31
cctccttactttgctgacagaaataatttg

SEQ ID NO: 38 - DNA, 28 bp, Artificial Sequence
  P32
gtcagcaaagtaaggaggttccgtacac

SEQ ID NO: 39 - protein, 17 aa, Mouse
YIYPYTGGTGYSQKFKS

SEQ ID NO: 40 - protein, 17 aa, Homo sapiens
WMNPNSGNTGYAQKFQG

SEQ ID NO: 41 - protein, 8 aa, Mouse
NYGYDESA

SEQ ID NO: 42 - protein, 112 aa, Homo sapiens HzS-III-VL
DIVLTQSPASLAVSPGQRATITCRASESVSNYGINFINWFQQKPGQPPKLLIYTASNKGTGVPARFSGSGSGTDFTLTINPVEAEDTANYFCQQSKEVPYTFGGGTKVEIKR

SEQ ID NO: 43 - protein, 118 aa, Homo sapiens HzS-III-VH
EVQLVQSGAEVKKPGASVKVSCKASGYTFTDYNVQWVRQAPGKSLEWMGYIYPYSGGTGYSQKFQGRVTLTVDNSASTAYMELSSLRSEDTAVYYCARNYGYDSSAYWGQGTLVTVSS

SEQ ID NO: 44 - protein, 9 aa, Artificial Sequence
  peptide sequence which binds to MHC class II molecule
WVRQAPGKS

SEQ ID NO: 45 - protein, 9 aa, Artificial Sequence
  peptide sequence which binds to MHC class II molecule
FQGRVTLTV

SEQ ID NO: 46 - DNA, 23 bp, Artificial Sequence
  P33
ggaaagacccttgagtggatggg

SEQ ID NO: 47 - DNA, 22 bp, Artificial Sequence
  P34
ctcaagggtctttccgggggcc

SEQ ID NO: 48 - DNA, 28 bp, Artificial Sequence
  P35
gcagagtcgatttgactgtagacaattc

SEQ ID NO: 49 - DNA, 26 bp, Artificial Sequence
  P36
cagtcaaatcgactctgccctggaac

SEQ ID NO: 50 - protein, 9 aa, Artificial Sequence
  peptide sequence which binds to MHC class II molecule
WVRQAPGKT

SEQ ID NO: 51 - protein, 9 aa, Artificial Sequence
  peptide sequence which binds to MHC class II molecule
FQGRVDLTV

SEQ ID NO: 52 - protein, 118 aa, Homo sapiens HzS-IV-VH
EVQLVQSGAEVKKPGASVKVSCKASGYTFTDYNVQWVRQAPGKTLEWMGYIYPYSGGTGYSQKFQGRVDLTVDNSASTAYMELSSLRSEDTAVYYCARNYGYDSSGYWGQGTLVTVSS